(19) United States
(12) Patent Application Publication
(10) Pub. No.: US 2024/0038335 A1
(43) Pub. Date: Feb. 1, 2024

(54) SYSTEMS AND METHODS FOR DETECTING DISEASE SUBTYPES

(71) Applicant: GRAIL, LLC, Menlo Park, CA (US)

(72) Inventors: Tracy NANCE, Menlo Park, CA (US); Joerg BREDNO, San Francisco, CA (US); Oliver Claude VENN, San Francisco, CA (US); Robert Abe Paine CALEF, Redwood City, CA (US); Jennifer TOM, Hillsborough, CA (US)

(73) Assignee: GRAIL, LLC, Menlo Park, CA (US)

(21) Appl. No.: 18/362,342

(22) Filed: Jul. 31, 2023

Related U.S. Application Data
(60) Provisional application No. 63/370,014, filed on Aug. 1, 2022.
(60) Provisional application No. 63/382,664, filed on Nov. 7, 2022.
(60) Provisional application No. 63/385,590, filed on Nov. 30, 2022.

(51) Int. Cl. G16B 30/00; G16B 40/20; G16H 10/40; G16H 50/50; G16H 50/20

(52) U.S. Cl. CPC G16B 30/00; G16B 40/20; G16H 10/40; G16H 50/50; G16H 50/20; G16H 50/30

(57) ABSTRACT

Systems and methods for detecting a subtype of a disease state and for determining the development of a resistance mechanism in a disease are disclosed. One method may include: receiving, at an input component of the system, a set of sequence reads associated with a nucleic acid sample; generating, using a processor of the system and via analysis of the set of sequence reads, methylation data; and analyzing, using the processor, the methylation data to identify the subtype of the disease state. Another method may include: obtaining methylation data from a targeted methylation sequencing assay, applying the methylation data to a trained machine learning model, and receiving an output indicating whether MRD is present in a test subject and/or whether a resistance mechanism has been developed by a disease. Other aspects are described and claimed.

### CROSS-REFERENCE TO RELATED APPLICATIONS

This application claims the benefit of priority to U.S. Provisional Patent Application No. 63/370,014, filed Aug. 1, 2022, U.S. Provisional Patent Application No. 63/382,664, filed Nov. 7, 2022, and U.S. Provisional Patent Application No. 63/385,590, filed Nov. 30, 2022, each of which is hereby incorporated by reference herein in its entirety.

### TECHNICAL FIELD

The present disclosure relates generally to the field of model-based featurization and classifiers for predicting disease state and subtypes from nucleic acid samples.

### BACKGROUND

It has been observed that Deoxyribonucleic acid (DNA) methylation plays an important role in regulating gene expression. Aberrant DNA methylation has been implicated in many disease processes, including cancer. Advances in research and technology have led to the development of new techniques for detecting various disease states at an earlier stage. For instance, DNA methylation profiling using methylation sequencing (e.g., whole genome bisulfite sequencing (WGBS)) is increasingly recognized as a valuable diagnostic tool for detection, diagnosis, and/or monitoring of cancer. More particularly, specific patterns of differentially methylated regions and/or allele specific methylation patterns may be useful as molecular markers for non-invasive diagnostics using circulating cell-free (cf) DNA. However, there remains a need in the art for improved methods for analyzing methylation sequencing data from cell-free DNA for the detection, diagnosis, and/or monitoring of diseases, such as cancer.

The present disclosure is directed to addressing one or more of these above-referenced challenges. The background description provided herein is for the purpose of generally presenting context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

In summary, one aspect provides a method of detecting a subtype of a disease state using a system, the method including: receiving, at an input component of the system, a set of sequence reads associated with a nucleic acid sample; generating, using a processor of the system and via analysis of the set of sequence reads, methylation data; and analyzing, using the processor, the methylation data to identify the subtype of the disease state.

Another aspect provides a method of training a machine learning model to detect a development of a resistance mechanism in a cancer undergoing a treatment, the method including: obtaining, from a source, a set of training data, wherein the training data comprises methylation data derived from a targeted methylation sequencing assay; annotating, subsequent to the obtaining, the set of training data by assigning a histologic label to each article of training data in the set; applying the annotated set of training data to the machine learning model; and optimizing, based on the applying, a pattern recognition capability of an algorithm associated with the machine learning model.

Yet another aspect provides a method of detecting a development of a resistance mechanism in a cancer undergoing a treatment using a trained machine learning model associated with a computer system, the method including: receiving, from a biological sample associated with a test subject, methylation data derived from a targeted methylation sequencing assay; applying, subsequent to the receiving, the methylation data to the trained machine learning model; and receiving, subsequent to the applying, an output from the trained machine learning model, the output comprising: A) a first indication of whether minimal residual disease is present within the test subject subsequent to administration of the treatment for the cancer; and B) a second indication, responsive to the first indication providing a finding that the minimal residual disease is present within the test subject, of whether at least a portion of cancer cells in the minimal residual disease have transformed from a first cancer type to a second cancer type as a result of the development of the resistance mechanism.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE DRAWINGS

The file of this patent contains at least one drawing/photograph executed in color. Copies of this patent with color drawing(s)/photograph(s) will be provided by the Office upon request and payment of the necessary fee.

The accompanying drawings, which are incorporated in and constitute a part of this specification, illustrate several embodiments and together with the description, serve to explain the principles of the disclosure.

FIG. 1A depicts an exemplary computer system for executing the techniques described herein.

FIG. 1B depicts an exemplary software platform for executing the techniques described herein.

FIG. 2 depicts a confusion matrix, according to one aspect of the present disclosure.

FIG. 3 depicts a table, according to one aspect of the present disclosure.

FIG. 4 depicts another table, according to one aspect of the present disclosure.

FIG. 5 depicts a heatmap, according to one aspect of the present disclosure.

FIG. 6A depicts a data plot, according to one aspect of the present disclosure.

FIG. 6B depicts a confusion matrix, according to one aspect of the present disclosure.

FIG. 7A depicts a heatmap, according to one aspect of the present disclosure.

FIG. 7B depicts another heatmap, according to one aspect of the present disclosure.

FIG. 8 depicts a bar graph, according to one aspect of the present disclosure.

FIG. 9A depicts a gene body illustration of a transcription factor, according to one aspect of the present disclosure.

FIG. 9B depicts another gene body illustration of a transcription factor motif, according to one aspect of the present disclosure.

FIG. 10A depicts a testing process flow, according to one aspect of the present disclosure.

FIG. 10B depicts another testing process flow, according to one aspect of the present disclosure.

FIG. 11 depicts an illustration of how beta values are calculated, according to one aspect of the present disclosure.

FIG. 12 depicts another heat map, according to one aspect of the present disclosure.

FIGS. 13A-F depict clustering graph patterns, according to one aspect of the present disclosure.

FIG. 14A depicts a line graph for a transcription factor, according to one aspect of the present disclosure.

FIG. 14B depicts a heatmap, according to one aspect of the present disclosure.

FIG. 15 depicts another string plot graph for a transcription factor, according to one aspect of the present disclosure.

FIG. 16 depicts another string plot graph for a transcription factor, according to one aspect of the present disclosure.

FIG. 17 depicts another string plot graph for a transcription factor, according to one aspect of the present disclosure.

FIG. 18 depicts another string plot graph for a transcription factor, according to one aspect of the present disclosure.

FIG. 19 depicts another heatmap, according to one aspect of the present disclosure.

FIG. 20A-F depict clustering graph patterns, according to one aspect of the present disclosure.

FIG. 21 depicts a graph of sample size considerations, according to one aspect of the present disclosure.

FIG. 22 depicts illustrative information, according to one aspect of the present disclosure.

FIG. 23 depicts illustrative information, according to one aspect of the present disclosure.

FIG. 24 depicts an exemplary method of training a machine learning model to determine whether a resistance mechanism has been developed in a cancer, according to one aspect of the present disclosure.

FIG. 25 depicts an exemplary method of utilizing a trained machine learning model to determine whether a resistance mechanism has been developed in a cancer, according to one aspect of the present disclosure.

FIG. 26 depicts a confusion matrix, according to one aspect of the present disclosure.

FIG. 27 depicts an exemplary method of training a machine learning model to generate a prognosis score from patterns in DNA methylation data, according to one aspect of the present disclosure.

FIG. 28 depicts illustrative information, according to one aspect of the present disclosure.

FIG. 29 depicts an exemplary method of generating a final patient prognosis score by combining a machine-generated prognosis score from a trained machine learning model with a ctDNA score, according to one aspect of the present disclosure.

FIG. 30 depicts a graph presenting patient prognosis scores generated from different approaches, according to one aspect of the present disclosure.

### DETAILED DESCRIPTION OF EMBODIMENTS

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. Relative terms, such as “about,” “approximately,” “substantially,” and “generally,” are used to indicate a possible variation of ±10% of a stated or understood value. In addition, the term “between” used in describing ranges of values is intended to include the minimum and maximum values described herein. The use of the term “or” in the claims and specification is used to mean “and/or” unless explicitly indicated to refer to alternatives only or the alternatives are mutually exclusive, although the disclosure supports a definition that refers to only alternatives and “and/or.” As used herein “another” may mean at least a second or more.

As used herein, the term “user” generally encompasses any person or entity, such as a researcher and/or a care provider (e.g., a doctor, etc.), that may desire information, resolution of an issue, or engage in any other type of interaction with a provider of the systems and methods described herein (e.g., via an application interface resident on their electronic device, etc.). The term “electronic application” or “application” may be used interchangeably with other terms like “program,” or the like, and generally encompasses software that is configured to interact with, modify, override, supplement, or operate in conjunction with other software.

As used herein, a “machine-learning model” generally encompasses instructions, data, and/or a model configured to receive input, and apply one or more of a weight, bias, classification, or analysis on the input to generate an output. The output may include, for example, an analysis based on the input, a prediction, suggestion, or recommendation associated with the input, a dynamic action performed by a system, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

DNA methylation has long been regarded as a hallmark of cancer and holds great promise for early-stage cancer detection. In particular, through the utilization of targeted whole genome bisulfite sequencing (“WGBS”), coupled with the processing capabilities associated with machine learning technologies, methylated DNA sequences can be effectively read and abnormally methylated sequences (i.e., those that may be indicative of cancer) may be identified. Accordingly, targeted DNA methylation assays performed on cell-free DNA (“cfDNA”) fragments may be capable of detecting multiple cancers across all stages, including at early stages when treatment may be more effective.

GRAIL's multi-cancer early detection (MCED) test uses a targeted methylation assay from a plasma sample, together with a trained classifier, to detect the presence of an invasive cancer and to identify the cancer signal origin in the patient's body (i.e., the “Tissue of Origin”). Furthermore, GRAIL's post-diagnostic program provides a non-tissue-informed estimate of circulating methylation variant allele frequency (mVaF) (i.e., circulating tumor fraction) to quantify disease burden and to detect minimal residual disease (MRD). Additionally, GRAIL has shown an ability to distinguish subtypes of different cancer types from cfDNA. More particularly, techniques have been developed that may leverage cfDNA methylation patterns to distinguish cancers broadly by embryologic origin of cancer and more specifically to separate epithelial solid cancers into histologic types or subtypes like adenocarcinoma, HPV-associated squamous cell carcinoma, non-HPV-associated squamous cell carcinoma, carcinoma of Mullerian origin, and transitional cell carcinoma, for example. GRAIL is further able to use cfDNA methylation patterns to distinguish Ovarian type II and Uterine high-grade serous carcinomas (a more aggressive subtype) from Ovarian type I, endometrioid and endometrial uterine cancers (a less aggressive subtype).

Embodiments of this application may utilize existing systems and processes to identify subtypes of cancer, as further described herein. Embodiments of the disclosure are drawn to the use of methylation data to define molecular subtypes of cancers generally, and the disclosure provides an in-depth case study of defining subtypes of small cell lung cancer (SCLC) as an example cancer.

SCLC is an aggressive form of lung cancer often accompanied by poor prognosis. In contrast to non-small cell lung cancers (NSCLC), for which therapies targeted to genomic mutations in diverse oncogenes have been deployed effectively, SCLC has not benefited from the targeted treatments, in part because SCLC malignancies are almost universally driven by loss-of-function mutations in the tumor suppressor genes TP53 and RB1.

Subtypes of SCLC have been identified using gene expression profiling, with four main subtypes being defined based on the expression levels of three key transcription factors: ASCL1, NEUROD1, and POU2F3.

Surgical resection is not frequently performed on late stage SCLC patients and SCLC is generally not detected until later stages. As a result, there may be limited access to tumor tissue. However, a blood-based methylation assay may have advantages in both research and development and clinical contexts. A methylation-based approach in cfDNA has been contemplated for the classification of lung cancer into broader types, namely NSCLC (and its subtypes: lung adenocarcinoma, lung squamous cell carcinoma, and large cell carcinoma) versus SCLC. However, such an approach may not identify methods for the more specific subtyping of SCLC into its component subtypes. Additionally such an approach focuses on methylation levels derived from quantitative methylation-specific PCR in only four prespecified genes (APC, HOXA9, RARB2, and RASSF1A), which do not include the three SCLC-subtype defining transcription factors defined in recent literature.

Accordingly, some aspects of the present disclosure are directed toward the utilization of a single targeted, blood-based methylation assay for early detection of cancer, quantification of disease burden for patients on treatment, and subtyping of cancers, e.g., SCLC. The identification of cancer molecular subtypes may be useful, for example, in understanding cancers, determining patient prognoses, or in guiding treatment decisions. More particularly, machine learning techniques may be employed on targeted methylation data from blood-derived cfDNA to identify molecular cancer subtypes and predict susceptibility of an individual cancer to therapeutic interventions. The embodiments disclosed herein propose to learn methylation signatures of cancer subtypes across different and potentially many more genomic loci than conventionally covered. Furthermore, the cancer subtyping with information for therapy selection could be performed with the same blood draw and sample that was used to detect the presence of invasive cancer in a screening setting. Although some embodiments of the disclosure are drawn to SCLC, the methods described herein may be used to identify molecular subtypes of a variety of cancers using methylation data.

Another situation addressed by some embodiments of the present disclosure relates to the detection of resistance mechanisms developed by some cancers under treatment. More particularly, a variety of targeted therapies exist and are available to treat many frequent types of cancer, such as adenocarcinomas. For instance, epidermal growth factor receptor (EGFR) mutated lung adenocarcinomas are potentially treated with EGFR inhibition and prostate cancers are potentially treated with hormone deprivation therapy or androgen receptor signal inhibition.

Cancers under treatment-related selection pressure may develop certain resistance mechanisms. For example, lung adenocarcinomas under EGFR inhibition may show resistance after a period of time, e.g., on the order of about 12 months. As another example, prostate cancers may evade treatment and transform into castrate-resistant and potentially metastatic cancers. One mechanism of acquired treatment resistance is transdifferentiation of the tumor, i.e., the transformation of the original adenocarcinoma into a small cell neuroendocrine carcinoma. After transformation, the neuroendocrine carcinoma may no longer respond to the original therapy and may require selection of and implementation of a new treatment regimen. This type of treatment evasion has been reported in approximately 15-20% of late stage prostate cancers and 5-14% of adenocarcinomas that are resistant to EGFR inhibition.

Conventionally, transdifferentiation is typically only detected after the initial treatment has failed (i.e., as observed by a reccurrence of the tumor) and via a re-biopsy of a metastasis or a recurred or growing primary cancer. Re-biopsy may be a clinically intensive process, and by the time a re-biopsy is performed, it may generally be too late for a successful treatment adjustment. Solutions have been proposed to utilize genomic profiling with a target small variant assay to detect transdifferentiation from ctDNA. However, there may be too many shared genomic aberrations between the original adenocarcinoma and the transformed small cell carcinoma to identify a clear distinction between the respective genomic variants. For instance, mutations on the p53 and Rb1 pathways that are nearly ubiquitous in small cell carcinomas are also seen in adenocarcinomas and are not specific to a neuroendocrine transformation.

Treatment monitoring often includes disease burden assessment or detection of MRD, which corresponds to the small number of cancer cells that can remain in the body after treatment (e.g., tumor removal, therapy administration, etc.). During or after cancer treatment, any remaining cancer cells may become active and start to multiply, potentially resulting in a relapse of the disease. Accordingly, detecting MRD may indicate that a treatment was not completely effective or that the treatment was incomplete. MRD may be present, for instance, because certain cancer cells became resistant, e.g., via transdifferentiation, to the medications used. MRD may be detected, for example, via a liquid biopsy. However, even with a liquid biopsy assay available to detect transdifferentiation to small cell carcinoma, a second assay and analysis would be needed to obtain information on MRD and disease burden and possible transdifferentiation for disease monitoring, which may be time-consuming and/or burdensome.

Accordingly, in view of the foregoing, some aspects of the present disclosure are directed toward simultaneously leveraging the single, targeted, liquid biopsy (e.g., blood-based) methylation assay for both MRD assessment, as previously described above, to perform parallel disease monitoring, and to identify recurrence and/or development of a resistance mechanism in a cancer, e.g., via transdifferentiation. In this regard, machine learning techniques may be employed to recognize and differentiate between cancer types, e.g., adenocarcinoma and neuroendocrine carcinoma, by training a model to recognize cancer signal origin (CSO) based on histologic type labeling, rather than on anatomic-site-based labeling. One or more downstream actions may thereafter be performed, e.g., alternative treatment suggestions, and the administration of a different treatment therapy.

Another situation addressed by some embodiments of the present disclosure relates to patient prognosis. More particularly, a patient prognosis may provide an estimate of the course and/or outcome of a cancer by assessing the risk of death for the patient and/or the disease progression of the cancer. Knowledge of a patient's prognosis may be valuable for clinical trial design and/or therapy selection, e.g., a more aggressive treatment may be prescribed for a patient having a worse prognosis as opposed to a more conservative treatment for a patient having a less severe prognosis.

Prognostic biomarkers are desired for the management of subjects identified as having cancer. Understanding a subject's prognosis at the cancer detection stage—and particularly for early cancer detection—may influence one or more of treatment regimens (including surgical or non-surgical), follow-up screening schedules, or other aspects of cancer and patient management. For example, in the case of Stage I NSCLC, a prognostic biomarker may identify subjects with a worse prognosis, and thus an increased risk profile, which may then justify neo-adjuvant therapy for that subject. A wide variety of biomarkers, including proteomic and genomic biomarkers, are available for patient prognosis. For instance, tumor fraction (mVAF) is a biomarker that may be an important prognostic feature. More particularly, increased tumor fraction may be associated with a host of biological occurrences known to indicate poor patient prognosis, e.g., increased tumor size, presence of tumor-involved lymph nodes, presence of distant metastases, increased metabolic and mitotic activity of tumor cells, and invasion of tumor into various biological structures (e.g., blood vessels, lymph vessels, adjacent structures, etc.).

Existing prognostic information, which is obtained from liquid biopsy, compresses the presence of ctDNA into a single metric or ctDNA score, namely tumor fraction, mVAF, or ctDNA concentration. Although this score may provide valuable insight into a subject's disease condition, it is largely binary in nature, e.g., large tumor fraction corresponds to a poor prognosis whereas low tumor fraction corresponds to a good prognosis. However, studies have shown that some subjects with relatively large tumor fraction still do relatively well and sometimes better than other patients who have comparatively lower tumor fraction. It may therefore be advantageous to leverage additional, patient-specific information to better capture the wide heterogeneity of cancers and their impact on prognosis, even when a ctDNA score is similar.

Accordingly, in view of the foregoing, some aspects of the present disclosure are directed toward utilizing DNA methylation features to improve the accuracy of subject prognosis. More particularly, in an embodiment, a supervised machine learning classifier may be trained on DNA methylation data that is labeled with known outcomes for each subject (e.g., where each label provides an indication of subject survival after X years along with an indication of disease progression, etc.). A prognostic score may be obtained from the foregoing classifier and may thereafter be combined with the ctDNA score to generate a final prognostic score for a subject. This final prognostic score may provide a more accurate subject prognosis indication than using cTF, VAF, or ctDNA concentration alone.

More particularly to the foregoing, circulating tumor fraction cTF or methylation variant allele frequency mVAF measure what fraction of the DNA molecules in a plasma sample are from tumor cells (cTF) and carry a methylation signal that is nearly unique to tumor cells (mVAF). The circulating tumor DNA molecules bring a methylation signal that, independent of the total amount captured in cTF or mVAF, can contain different methylation patterns that characterize the tumor and therefore allow for a prognosis. Classifiers can be trained to learn and recognize methylation signals that indicate good or poor prognosis. Accordingly, once two independent prognostic metrics are obtained (i.e., the amount (cTF or mVAF) of ctDNA and the methylation pattern that the ctDNA carries), these metrics can be combined into one final metric (e.g., via multiplying the two metrics by each other).

The subject matter of the present disclosure will now be described more fully hereinafter with reference to the accompanying drawings, which form a part hereof, and which show, by way of illustration, specific exemplary embodiments. An embodiment or implementation described herein as “exemplary” is not to be construed as preferred or advantageous, for example, over other embodiments or implementations; rather, it is intended to reflect or indicate that the embodiment(s) is/are “example” embodiment(s). Subject matter may be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware, or any combination thereof. The following detailed description is, therefore, not intended to be taken in a limiting sense.

Throughout the specification and claims, terms may have nuanced meanings suggested or implied in context beyond an explicitly stated meaning. Likewise, the phrase “in one embodiment” or “in some embodiments” as used herein does not necessarily refer to the same embodiment and the phrase “in another embodiment” as used herein does not necessarily refer to a different embodiment. It is intended, for example, that claimed subject matter include combinations of exemplary embodiments in whole or in part.

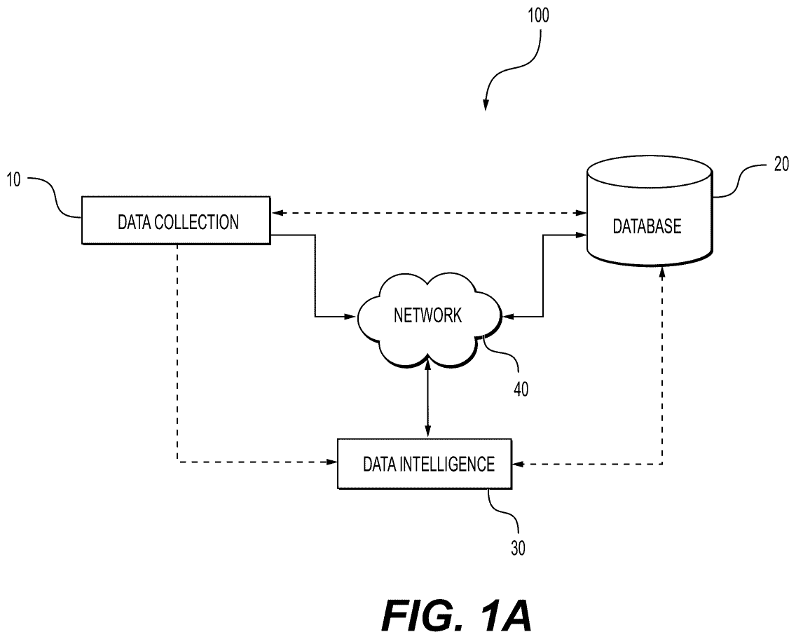

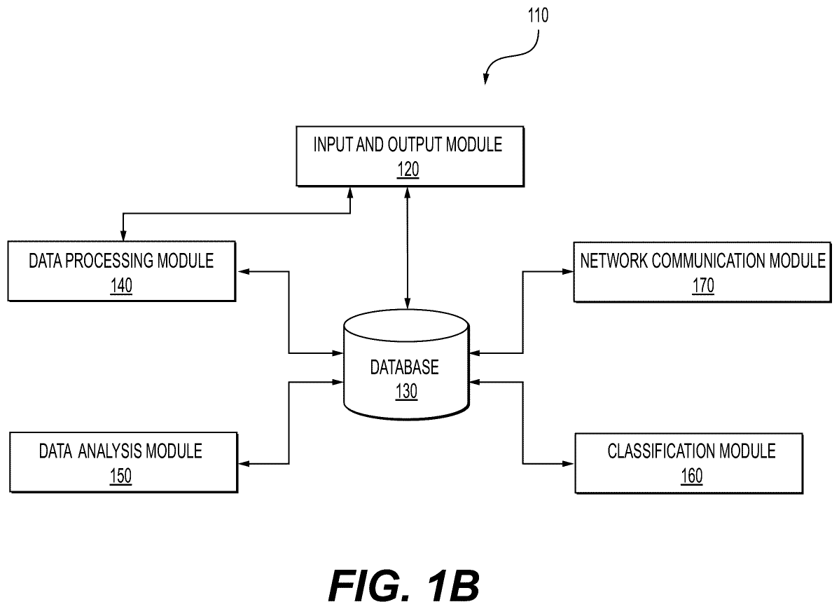

FIG. 1A depicts an exemplary system for utilizing a targeted methylation assay to identify cancer subtypes. Exemplary system 100 includes a data collection component 10, a database 20 and device data intelligence component 30, connected to each other via network 40. Alternatively, or additionally, one or more of the components may be connected with another component locally without reliance on network connection; e.g., through a wired connection. Sequencing data of cell-free nucleic acids are used to illustrate the concepts. However, one of skill in the art would understand that the current method may be applied to sequencing data of other materials or non-sequencing data as well.

As disclosed herein, data collection component 10 may include a device or machine with which sequencing data may be generated. In some embodiments, data collection component 10 may include a sequencing machine or a facility that uses a sequencing machine to generate nucleic acid sequence data of biological samples. Any applicable biological samples may be used. In some embodiments, a biological sample is cell-based; for example, one or more types of tissue. In some embodiments, a biological sample is a sample that includes cell-free nucleic acid fragments. Examples of biological samples include, but are not limited to, a blood sample, a serum sample, a plasma sample, a urine sample, a saliva sample, and etc.

Examples of sequencing data may include, but are not limited to, sequence read data of targeted genomic locations, partial or whole genome sequencing data of genome represented by nucleic acid fragments in cell-free or cell-based samples, partial or whole genome sequencing data including one or more types of epigenetic modifications (e.g., methylation), or combinations thereof.

Data acquired by the data collection component 10 may be transferred to database 20 via network 40. In some embodiments, the collected data may be analyzed by data intelligence component 30, via local or network connection. FIG. 1B depicts exemplary functional modules that may be implemented to perform tasks of data intelligence component 30.

FIG. 1B depicts an exemplary computer system 110 for processing sequencing data. Exemplary embodiment 110 achieves such functionalities by implementing, on one or more computer devices, user input and output (I/O) module 120, memory or database 130, data processing module 140, data analysis module 150, classification module 160, network communication module 170, and any other functional modules that may be needed for carrying out a particular task (e.g., an error correction or compensation module, a data compression module, and etc.). As disclosed herein, user I/O module 120 may further include an input sub-module, such as a keyboard, and an output sub-module, such as a display (e.g., a printer, a monitor, or a touchpad). In some embodiments, all functionalities are performed by one computer system. In some embodiments, the functionalities are performed by more than one computer.

Also disclosed herein, a particular task may be performed by implementing one or more functional modules. In particular, each of the enumerated modules itself may, in turn, include multiple sub-modules. For example, data processing module 140 may include a sub-module for data quality evaluation (e.g., for discarding very short sequence reads or sequence reads including obvious errors), a sub-module for normalizing numbers of sequence reads that align to different regions of a reference genome, a sub-module to compensate/correct GC biases, and etc.

In some embodiments, a user may use I/O module 120 to manipulate data that is available either on a local device or can be obtained via a network connection from a remote service device or another user device. For example, I/O module 120 may allow a user, e.g., via a keyboard, a mouse, or a touchpad, to perform data analysis via a graphical user interface (GUI). In some embodiments, a user may manipulate data via voice control. In some embodiments, user authentication may be required before a user is granted access to the data being requested.

In some embodiments, user I/O module 120 may be used to manage various functional modules. For example, a user may request via user I/O module 120 input data while an existing data processing session is in process. A user may do so by selecting a menu option or type in a command discretely without interrupting the existing process.

As disclosed herein, a user may use any type of input to direct and control data processing and analysis via I/O module 120.

In some embodiments, system 110 further comprises a memory or database 130. In some embodiments, database 130 comprises a local database that may be accessed via user I/O module 120. In some embodiments, database 130 comprises a remote database that may be accessed by user I/O module 120 via network connection. In some embodiments, database 130 is a local database that stores data retrieved from another device (e.g., a user device or a server). In some embodiments, memory or database 130 may store data retrieved in real-time from internet searches.

In some embodiments, database 130 may send data to and receive data from one or more of the other functional modules, including, but not limited to, a data collection module (not shown), data processing module 140, data analysis module 150, classification module 160, network communication module 170, and etc.

In some embodiments, database 130 may be a database local to the other functional modules. In some embodiments, database 130 may be a remote database that may be accessed by the other functional modules via wired or wireless network connection (e.g., via network communication module 170). In some embodiments, database 130 may include a local portion and a remote portion.

In some embodiments, system 110 comprises a data processing module 140. Data processing module 140 may receive the real-time data, from I/O module 120 or database 130. In some embodiments, data processing module 140 may perform standard data processing algorithms such as one or more of noise reduction, signal enhancement, normalization of counts of sequence reads, correction of GC bias, and etc. In some embodiments, data processing module 140 may identify global or local systematic errors. For example, sequencing data may be aligned to regions within a reference genome. The numbers of sequence reads aligned to different genomic regions may vary for the same subject. The numbers of sequence reads aligned to the same genomic regions may vary between subjects. Some of these differences, especially those observed in healthy subjects (i.e., including all types of organisms, not just humans), may result from systematic errors instead of an association with one or more diseased conditions. For example, if sequencing data corresponding to a particular genomic region shows wide ranges of variation between healthy subjects, data processing module 140 may classify the particular genomic region as a high-noise region and may exclude the corresponding data from further analysis. In some embodiments, the identification and treatment of possible systematic errors may be performed by data analysis module 140, as illustrated below.

In some embodiments, system 110 comprises a data analysis module 150. In some embodiments, data analysis module 150 includes identifying and treating systematic errors in sequencing data, as described in connection with data processing module 140.

In some embodiments, system 110 comprises a classification module 160, which analyzes data from a test subject whose status with respect to a medical condition is unknown and subsequently classifies the unknown test subject based on the likelihood of the subject fitting into a particular category. In some embodiments, the one or more parameters include a binomial probability score that is calculated based on logistic regression analysis. As disclosed herein, the binomial probability score may correspond to the likelihood of a subject having a certain medical condition such as cancer. For example, a score of over a predefined threshold may indicate the likelihood of having a cancer of a subtype of interest. In some embodiments, the one or more parameters may include a sequencing data distribution pattern correlating with the presence of a certain type or subtype of cancer. A subject with a pattern resembling the cancer pattern may be diagnosed as having cancer of this type. In some embodiments, a sequencing data distribution pattern may be identified in connection with more than one specific type of cancer, thus allowing an unknown subject to be classified with further details.

As disclosed herein, network communication module 170 may be used to facilitate communications between a user device, one or more databases, and any other suitable system or device through a wired or wireless network connection. Any communication protocol/device may be used, including without limitation a modem, an Ethernet connection, a network card (wireless or wired), an infrared communication device, a wireless communication device, and/or a chipset (such as a Bluetooth™ device, an 802.11 device, a WiFi device, a WiMax device, cellular communication facilities, etc.), a near-field communication (NFC), a Zigbee communication, a radio frequency (RF) or radio-frequency identification (RFID) communication, a PLC protocol, a 3G/4G/5G/LTE based communication, and/or the like. For example, a user device having a user interface platform for processing/analyzing low coverage sequencing data may communicate with another user device with the same platform, a regular user device without the same platform (e.g., a regular smartphone), a remote server, a physical device of a remote IoT local network, a wearable device, a user device communicably connected to a remote server, and etc.

The functional modules described herein are provided by way of example. It will be understood that different functional modules can be combined to create different utilities. It will also be understood that additional functional modules or sub-modules may be created to implement a certain utility.

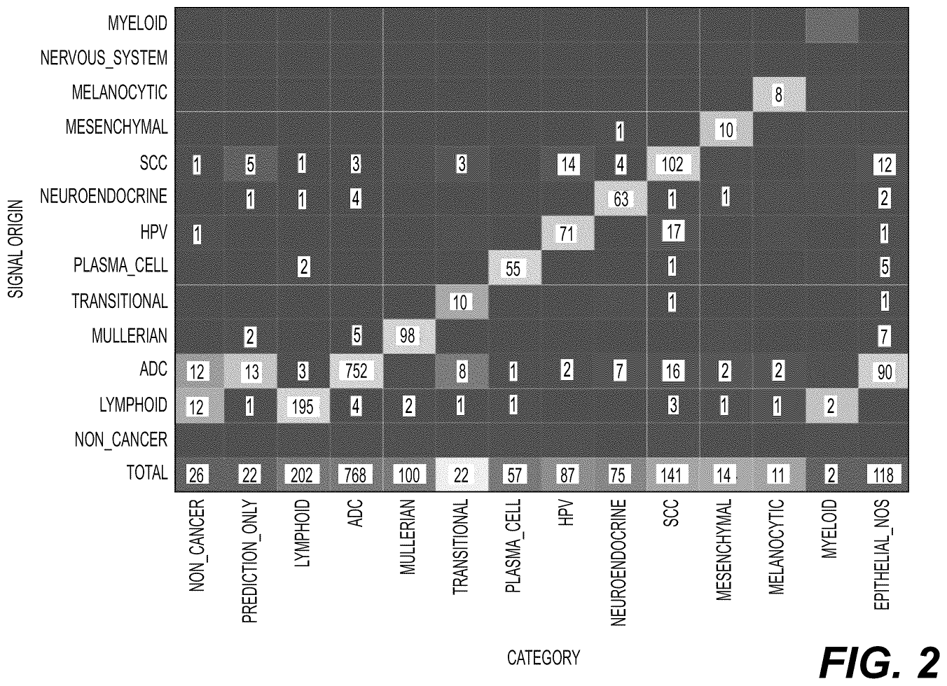

FIGS. 2-6 below illustrate how a trained cancer classifier can utilize cfDNA methylation data to identify subtypes of cancer.

Referring now to FIG. 2, a confusion matrix is provided that presents data, generated by a machine learning model (i.e., a trained cancer classifier), for samples identified as having a particular histologic type or cancer subtype. In an embodiment, a classifier using cfDNA methylation as input was trained to distinguish the embryologic lineage of cancer cells (epithelial, nervous system, lymphoid, myeloid, plasma cells, mesenchymal, melanocytic, neuroendocrine, and germ cells) and to further distinguish for all epithelial cancers (which is the most common type of solid cancer) their histologic types, namely adenocarcinoma (adc), HPV-associated squamous cell carcinoma (hpv), non-HPV-associated squamous cell carcinoma (scc), carcinoma of Mullerian origin (mullerian), and transitional cell carcinoma. The classifier was evaluated in cross-validation on a set of 2920 plasma samples from patients with cancer and 3704 plasma samples from patients without cancer. For 1619 samples, the plasma sample contained detectable levels of circulating tumor DNA (ctDNA).

In the confusion matrix illustrated in FIG. 2, each row contains the samples from one ground truth embryologic lineage and histologic type of cancer. Each column represents the samples with detectable ctDNA that this trained classifier identified as having this histologic type or cancer subtype. The samples counted on the diagonal represent the cases where the classifier correctly identified cancers by embryologic origin and histologic type based on the methylation signal in cfDNA.

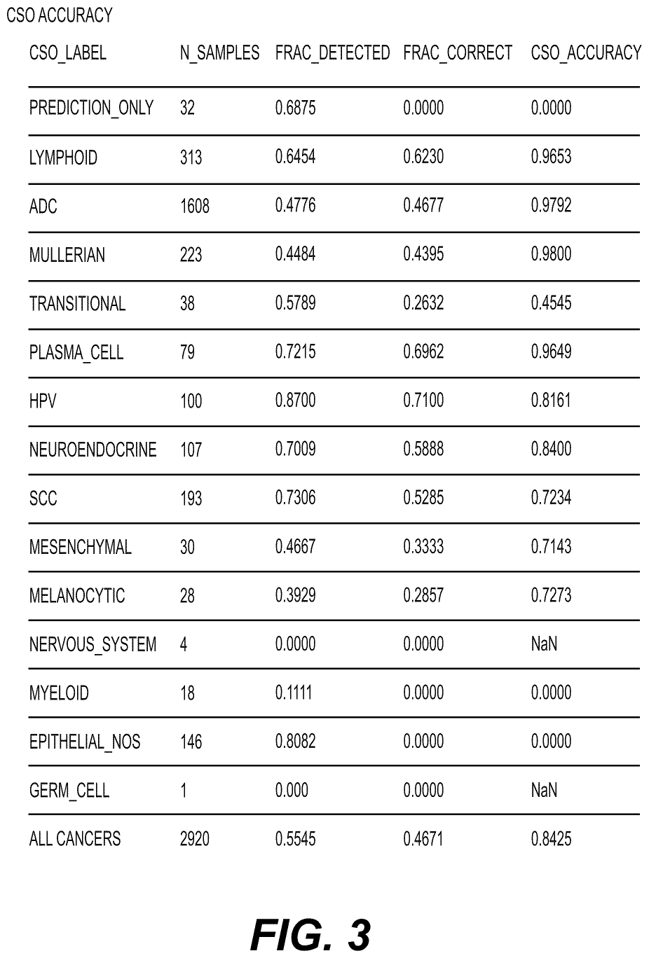

Referring now to FIG. 3, a table is presented that lists the accuracy (number of cases detected and number having the correct cancer signal origin result) broken down by each ground truth class.

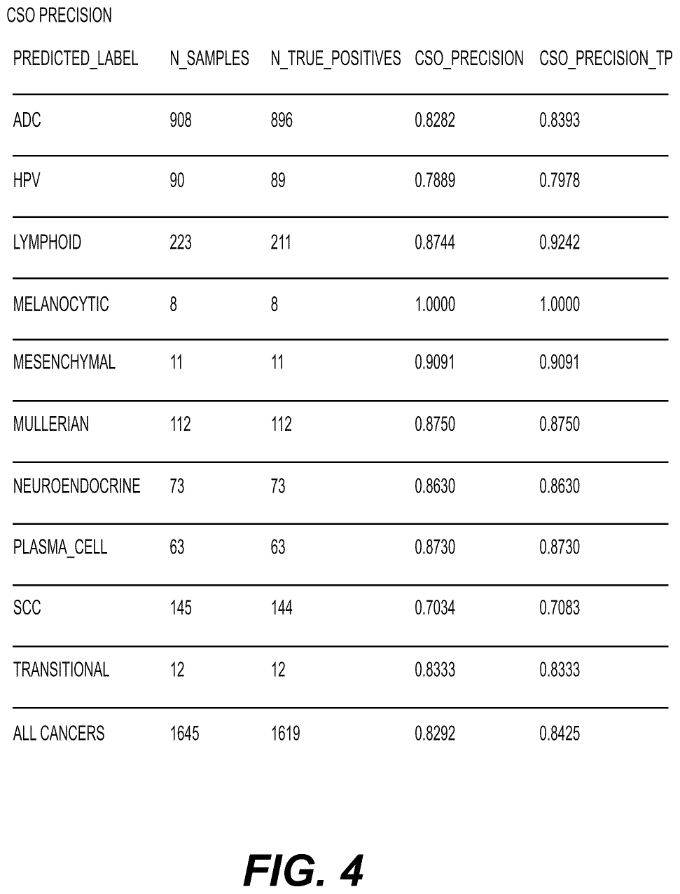

FIGS. 3 and 4 show a CSO accuracy of 84% and a CSO precision of 86.3% to identify neuroendocrine carcinomas and distinguish them from epithelial carcinomas. For lung cancers, this distinction allows the classifier to reliably separate Non-small cell lung cancer NSCLC from Small cell lung cancer SCLC using only a plasma sample obtained from a blood draw without the need to obtain a tissue sample from a lung cancer.

Referring now to FIG. 4, a table is presented that lists the precision of the classifier discussed in FIG. 2. For each class, it is listed how often a class returned by the classifier is correct and the sample actually has this class. The number is once presented for all cases detected by the classifier (cso_precision) and once limited to the cases that actually are an invasive cancer (cso_precision_tp). In this latter column, results from samples where the patient did not have cancer (false positive detection) are not included.

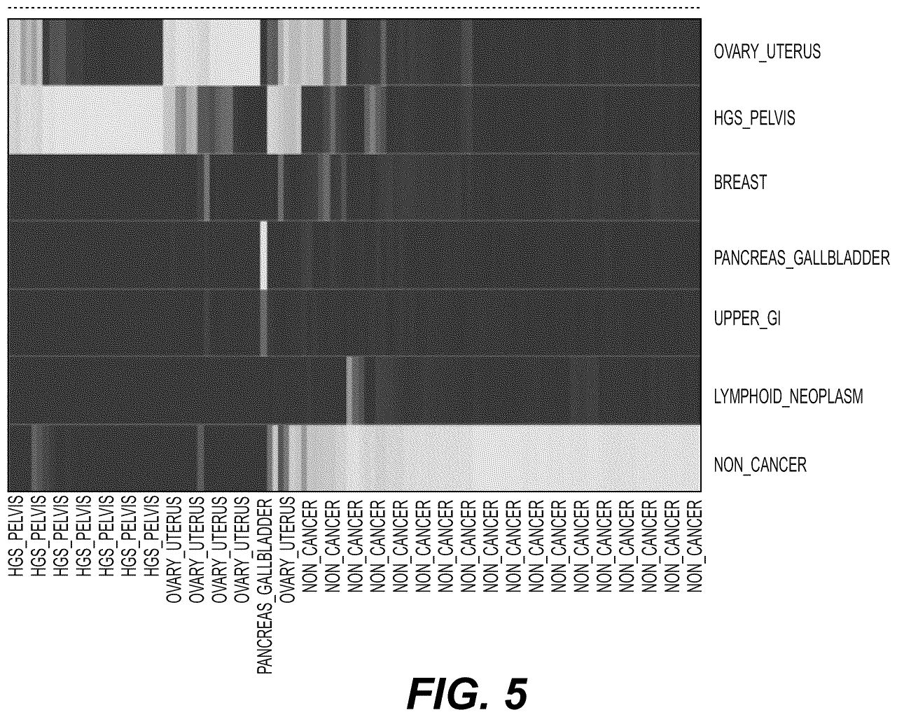

Referring now to FIG. 5, a heatmap is presented that shows a classifier result for cancers of the ovary and uterus having different histologic type and anatomic location of the primary cancer. In an embodiment, the instant classifier was trained with classes defined by anatomical origin and additionally training two different subtypes of ovarian and uterine cancers. More particularly, a class 1 ovarian cancer has its cell of origin in the epithelial cells of the ovaries. This is typically a slower growing cancer with a comparably good prognosis. A class 2 ovarian cancer is a cancer where the cell of origin is presumably in the Fallopian tubes. These neoplasms in the Fallopian tubes disseminate tumor cells very early (possibly from neoplastic growths of only a few hundred cells) and colonize the ovaries. These cancers are typically aggressive and nearly exclusively detected when they have already created metastases in the ovaries. These tumor metastases from Fallopian tube origin are also found in the uterus and the lining of the peritoneum. Together, type II ovarian cancers and these similar cancers of Fallopian tube origin are characterized as high-grade serous carcinomas.

In the classifier illustrated in FIG. 5, all high-grade serous carcinomas were trained as one class (i.e., named hgs_pelvis), and epithelial grade 1 or endometrial cancers of ovary and uterus were trained as a second class (i.e., named ovary_uterus). FIG. 5 shows for all cancers of the ovary and uterus the classifier result. More particularly, the color in the heatmap changes from low classifier signal (blue) to high classifier signal (yellow). The classifier result for each sample is displayed as a column label, and the scores are shown as row labels. High-grade serous type II cancers can be distinguished from lower grade epithelial type I cancers. One of the cases was misclassified as cancer of pancreas or gallbladder, and not all plasma samples had detectable ctDNA (classifier result non_cancer).

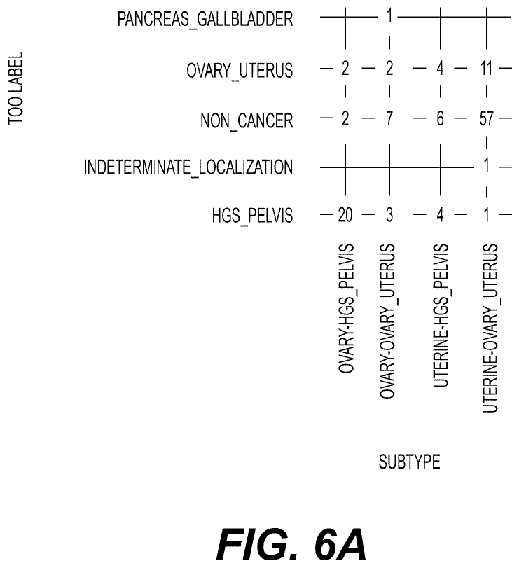

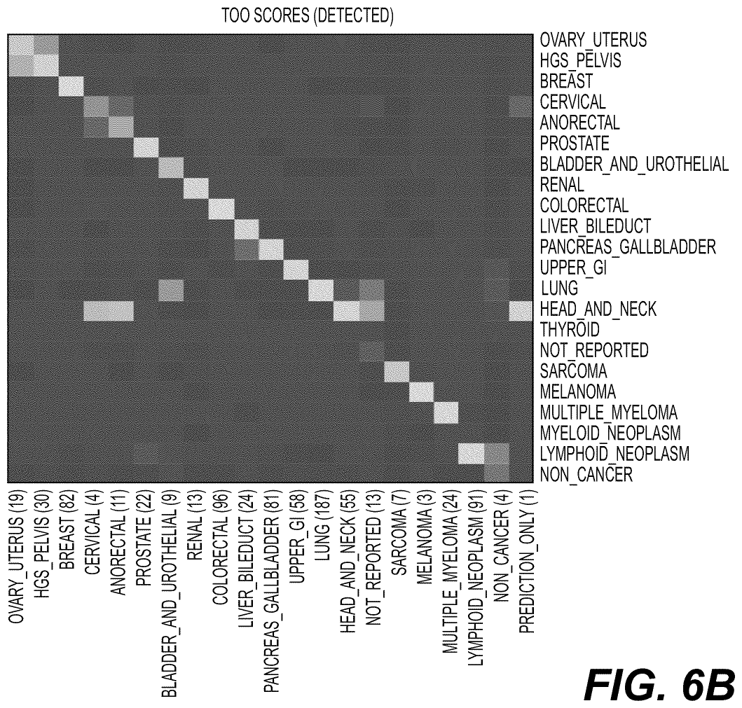

Referring now collectively to FIGS. 6A and 6B, a data plot and confusion matrix are provided that show the classifier result from FIG. 5 broken down by ground truth column labels, namely, high-grade serous type II cancers of ovary, epithelial type I cancers of ovary, high-grade serous cancers of the uterus, and epithelial endometrial cancers of the uterus. Type II high-grade serous cancers of the ovary are reliably identified by this classifier (20 cases ctDNA detected and correctly classified, 2 cases without detectable ctDNA, 2 cases with detectable ctDNA but misclassified as type I cancer).

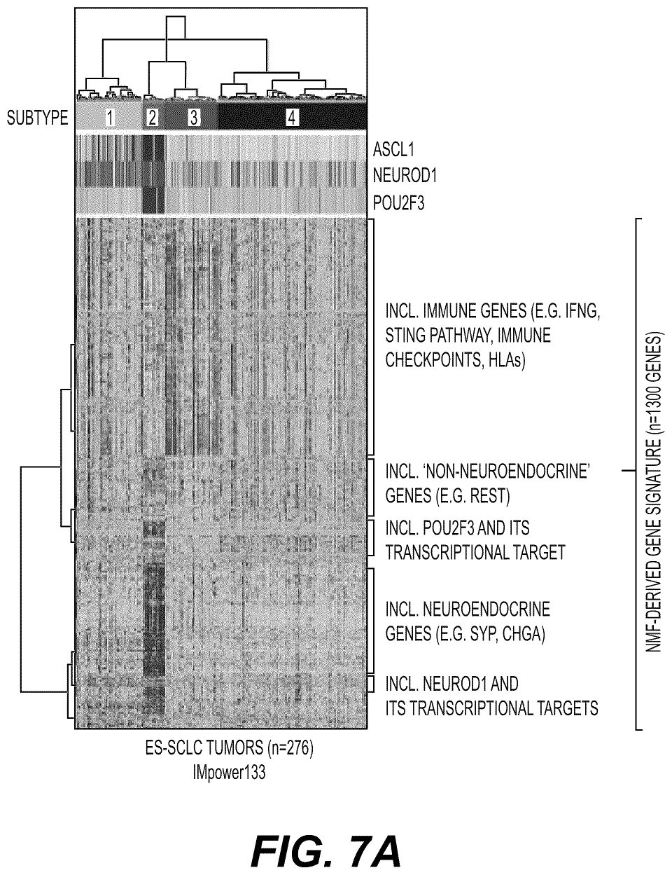

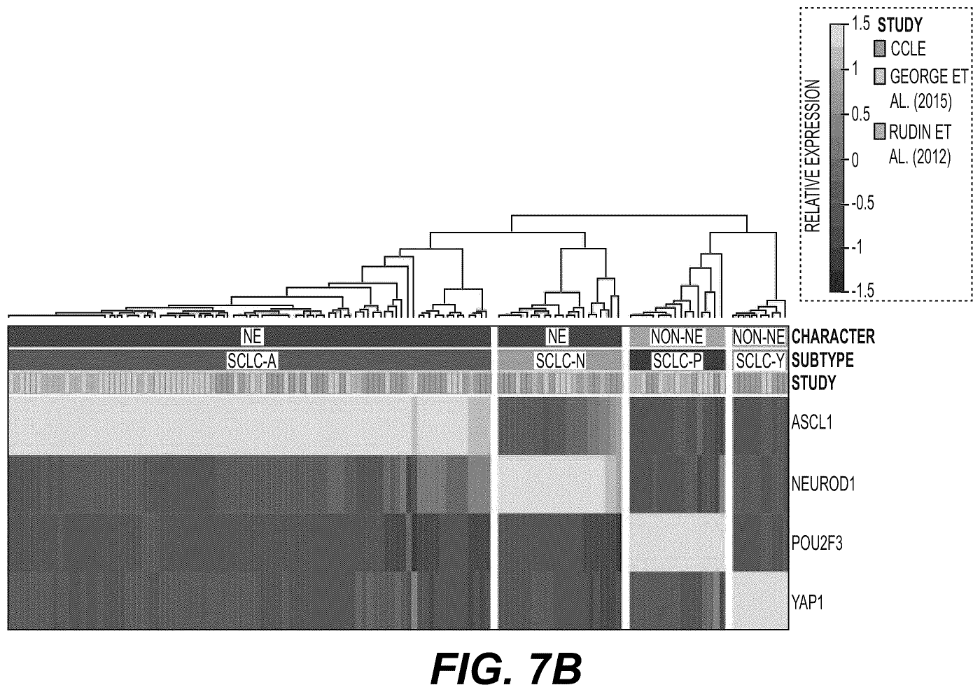

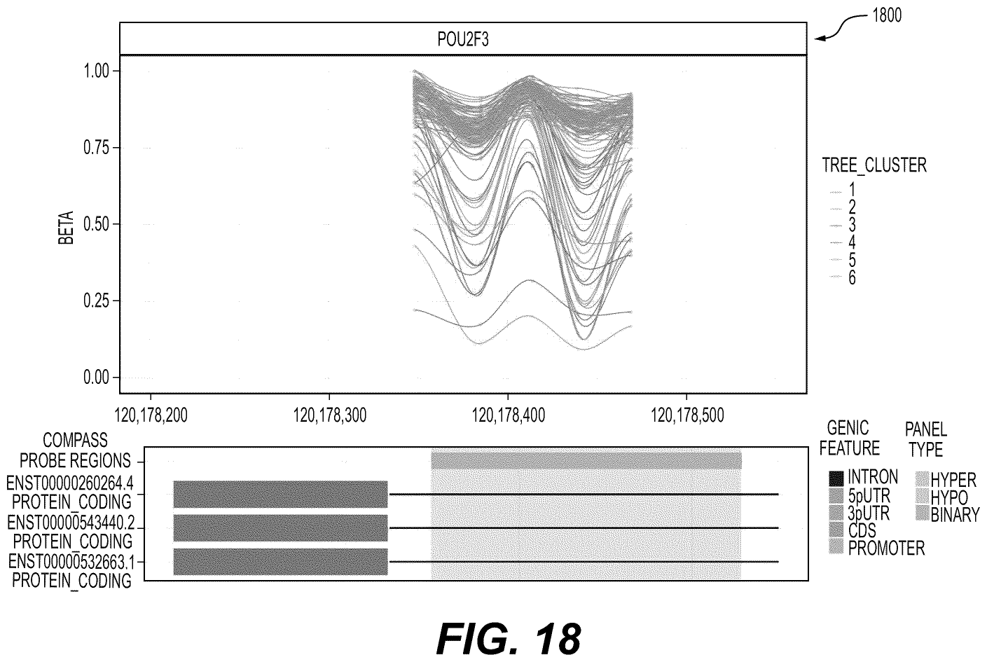

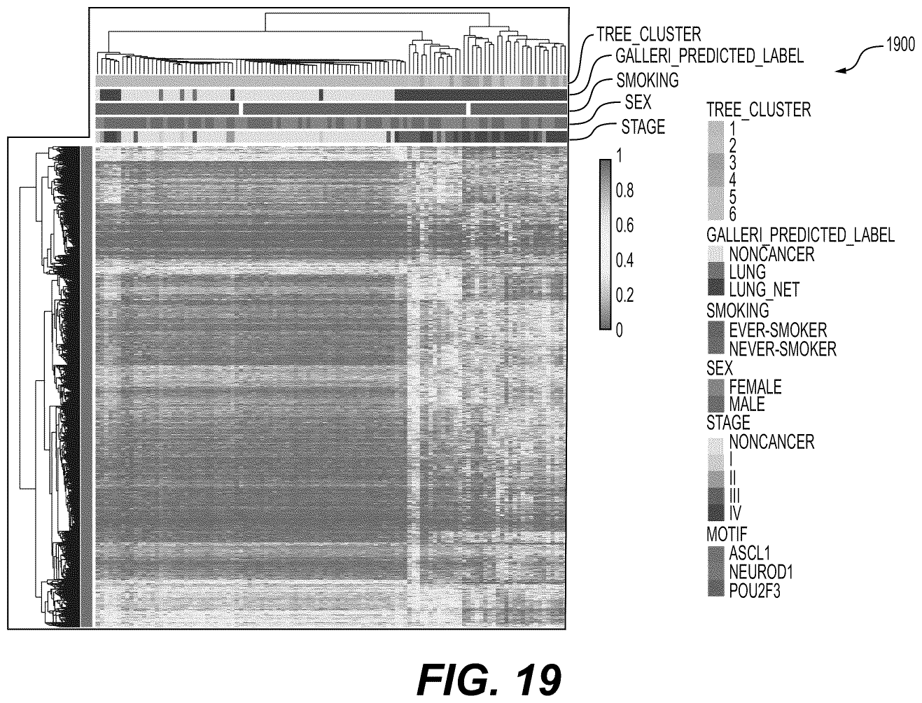

Some or all portions of the processes identified in FIGS. 2-6 above may be applicable to the identification of SCLC subtypes, as further described herein and illustrated in FIGS. 7-26. Additionally, the entire disclosures of commonly owned U.S. Patent App. Pub. No. 2020/0365229 and U.S. Patent App. Pub. No. 2021/0313006 are incorporated by reference herein except for any definitions, subject matter disclaimers or disavowals, and except to the extent that the incorporated material is inconsistent with the express disclosure herein, in which case the language in this disclosure controls. Some or all portions of U.S. Patent App. Pub. No. 2020/0365229 and U.S. Patent App. Pub. No. 2021/0313006 may also be applicable to the identification of cancer subtypes.

Referring now to FIGS. 7A and 7B, heatmaps of different known subtypes of SCLC that have been recorded in the literature previously are provided. These subtypes are currently being defined by expression signatures in tissue and, more particularly, are essentially defined by three transcription factors, i.e., the expression levels of three genes: ASCL1, NEUROD1, and POU2F3. The expression level of a fourth transcription factor, YAP1, may also have subtype implications. The SCLC subtypes in FIGS. 7A and 7B were identified using RNA sequencing on tissue samples and cell lines. Cancer tissue samples are often not available for the management of patients diagnosed with SCLC. It is therefore desired to identify these or other subtypes using only a plasma sample obtained with a simple blood draw. Additionally, an examination of the heatmaps in FIGS. 7A and 7B indicate that the expression levels of these genes are generally mutually exclusive in the tissue and cell line data presented therein. However, data generated from tissue samples may not reflect the intratumoral heterogeneity that may be detected from a plasma sample, which may give a fuller understanding of SCLC subtype heterogeneity and treatment implications.

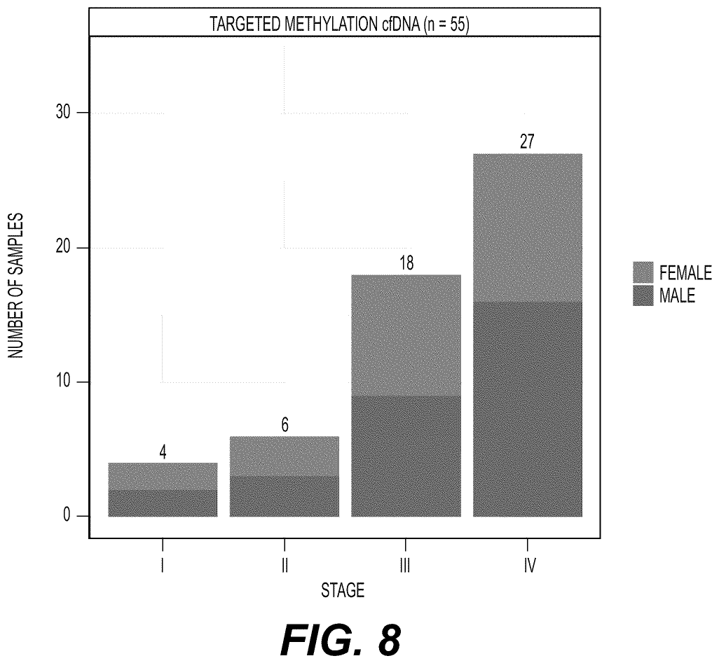

Referring now to FIG. 8, a bar graph is provided that illustrates the different types of samples that were obtained to conduct an analysis of methylation signatures for use in identifying SCLC subtypes. More particularly, samples were collected as part of a Circulating Cell-Free Genome Atlas (CCGA) Study in which 15,000 cancer and noncancer participants enrolled in a prospective, observational case-control study for training and validation of the Galleri classifier. Fifty-five patients with SCLC were selected for the analytical processes described herein with a breakdown by patient sex and clinical stage as shown in FIG. 8, as well as 57 non-cancer samples matched by age, sex, and smoking status.

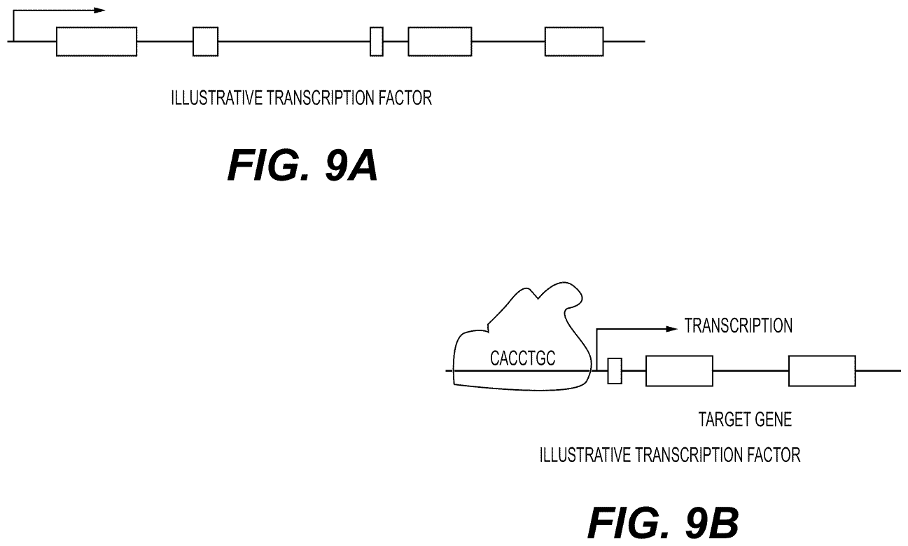

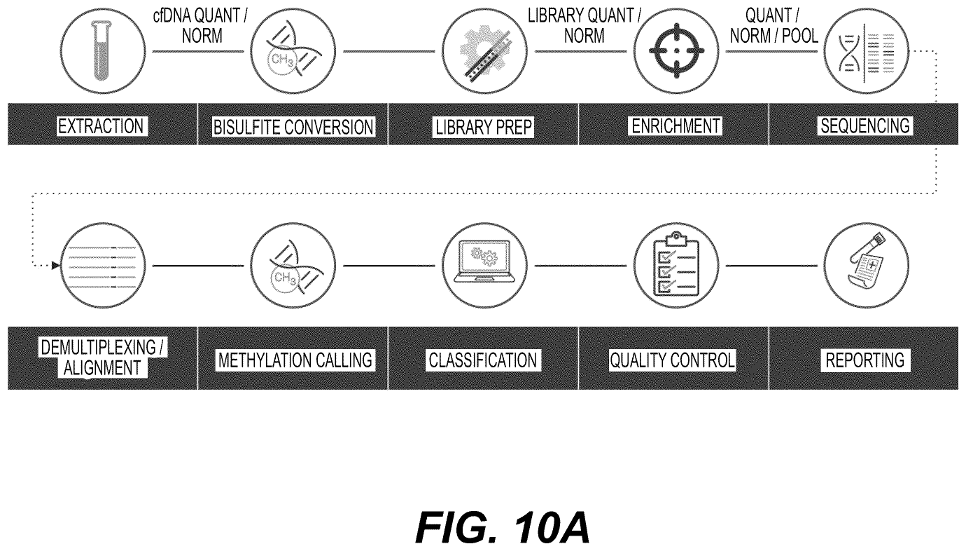

Referring now to FIGS. 9A and 9B, hypothetical gene body illustrations of a transcription factor gene body and motif are provided. One such hypothetical transcription factor may be, for example, ASCL1. Although no information presently exists about the response to therapy for the patients sampled (e.g., as illustrated in FIG. 8), methylation patterns may be examined in different regions of the genome that are biologically associated with subtype-defining transcription factors. More particularly, the transcription factors that define the subtypes code for proteins that bind to specific genomic sequences (i.e., motifs) that may be found throughout the genome. Through the examinations described herein, it may be shown that there is stratification in regions that have biological association with the known subtyping biomarkers. Specifically, substructures may be observed in the methylation signal that allow for the generation of the aforementioned subtypes using methylation data. One or all of these candidate subtypes may eventually be found to be predictive of a therapy response or prognostic of clinical outcomes.

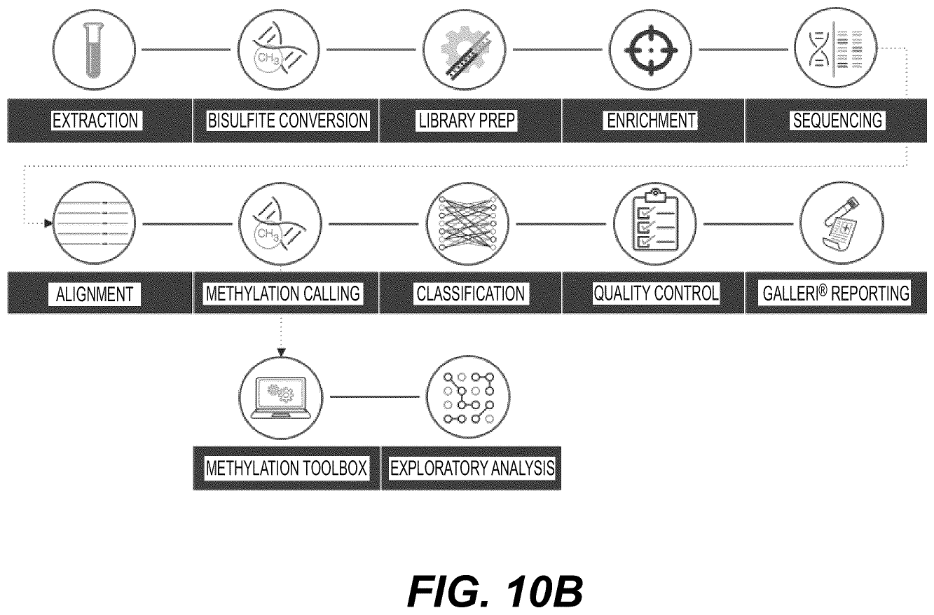

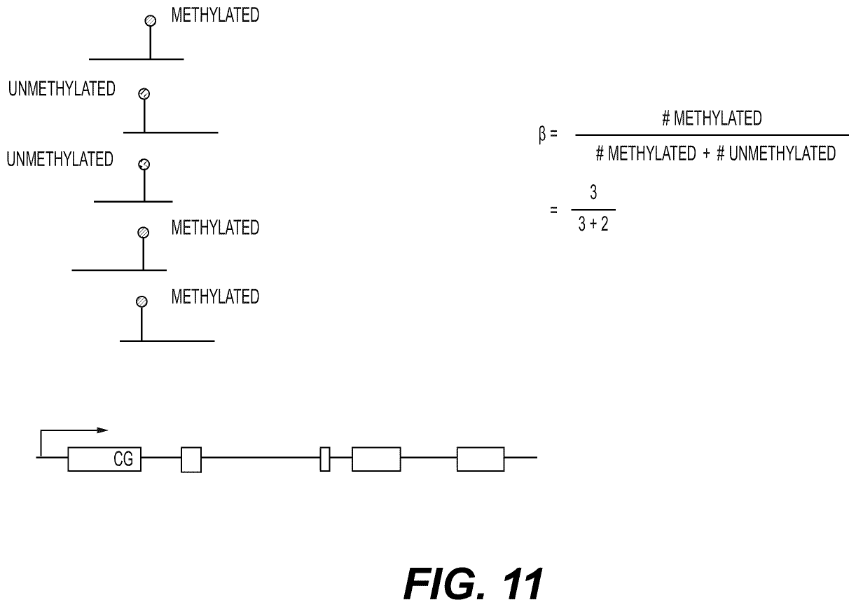

Referring now to FIG. 10A, a general flow chart of a cfDNA methylation testing process is provided. First a sample, e.g., a blood sample, may be collected from a subject. cfDNA in the sample may be evaluated through the use of bisulfite conversion, which may allow for distinguishing methylated versus unmethylated cytosines in the cfDNA. Library preparation may then be conducted through suitable means, and an enrichment step may be performed. Sequencing may then be performed, followed by demultiplexing and alignment, and then methylation calling. Based on the methylation data, classification of a cancer type, histologic type, or subtype may occur. Finally, quality control and reporting to a healthcare provider or patient may be performed. Embodiments of the disclosure may provide more data with which to perform the classification, or may allow for additional data to be output during the classification step.

A flow diagram of how exploratory analysis of methylation data may be incorporated into the current cfDNA methylation testing process is provided in FIG. 10B. As shown in FIG. 10B, following methylation calling, methylation data may be analyzed to identify a cancer subtype, and analysis may be performed based on the subtype of cancer identified. This information may be used to improve the classification step, or to broaden the scope of clinical or biological predictions that may be provided by the classifier. This methylation analysis step, referred to in FIG. 10B as the methylation toolbox, may allow for the exploration of methylation signature substructures on samples. In some aspects, analysis of methylation signature substructures may be performed on thousands of samples very quickly.

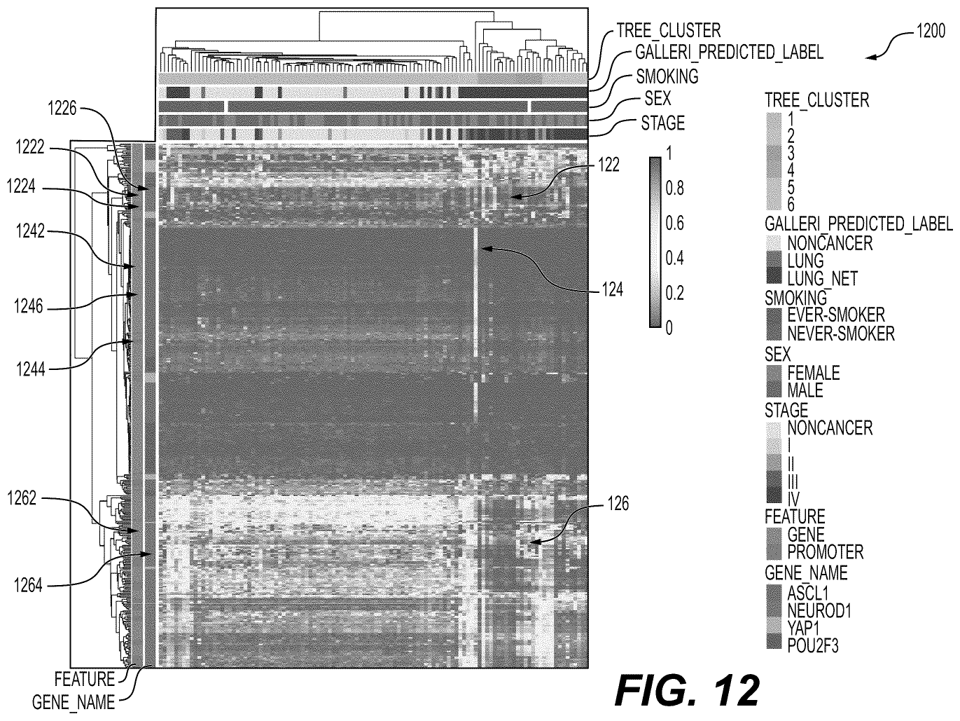

Referring now to FIG. 11, an illustration of how methylation beta values, or methylation-like beta values, are calculated is provided. Beta values refer to the fraction of molecules that are identified at particular CpG sites that are methylated. This fraction may be calculated by dividing the number of methylated molecules by the total number of molecules. For illustrative purposes, red represents methylated molecules and blue represents unmethylated molecules.

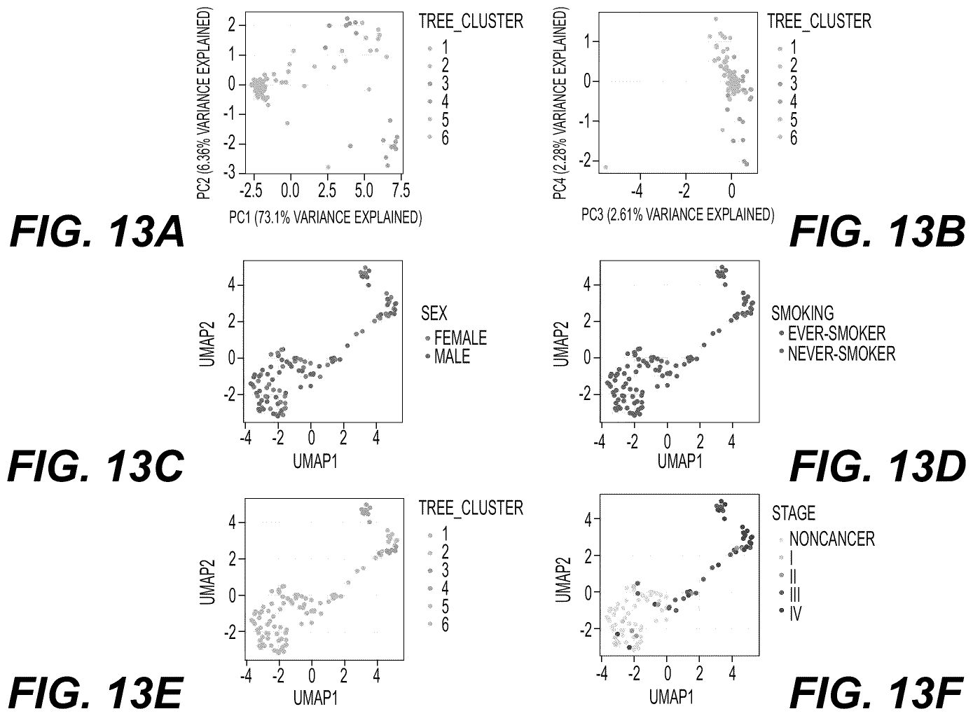

Referring now to FIG. 12, a heatmap 1200 illustrating methylation beta values per CpG in certain regions of the gene body and promoters of SCLC-relevant transcription factors is provided. The CpGs chosen are those lying in gene body and promoter regions of SCLC-subtype defining transcription factors, but alternative regions could be chosen, including one or more of: regions identified as being potential transcription factor binding sites, regions or individual CpG loci identified as differentially methylated between known, transcriptionally defined SCLC subtypes, regions or individual CpG loci identified as differentially methylated between responders and nonresponders to a particular therapy, and/or regions or individual CpG loci correlated with any of those defined above.

With reference to the heatmap 1200, CpGs are shown on rows whereas samples are shown on columns. The rows have been annotated to illustrate which gene each CpG lies in and whether it lies in the gene body or its promoter. Examination of the heatmap 1200 may reveal that “blockiness” exists in the heatmap structure in areas where the samples are hypermethylated or hypomethylated. For instance, the heatmap 1200 reveals that the cluster of samples 122 has hypermethylation along the gene body 1222 and promoter region 1224 of NEUROD1 1226. As another example, sample 124 has hypermethylation along the gene body 1242 and promoter regions 1244 of ASCL1 1246. In yet another example, sample 126 has hypomethylation along the gene body 1262 of NEUROD1 1264.

Embodiments of this disclosure aim to use the beta values in a matrix like this one as feature values to create a cancer subtype classifier—in this instance an SCLC subtype classifier—using the feature values in a set of labeled training samples to create a machine learning model (for example a penalized regression model, support vector machine, shallow neural network, etc.) to identify various SCLC subtypes, as defined either by traditional expression profiles or by response to treatment.

In addition to beta values as feature values for this classifier, the machine learning model of the present embodiments may also utilize: total counts of methylated and unmethylated molecules at each region or CpG locus and/or counts of abnormally methylated molecules at each region of CpG locus. Consequently, a single run of GRAIL's current targeted methylation assay can be applied either for early cancer detection or disease burden monitoring and can also be used in parallel for detection of cancer subtypes, e.g., SCLC subtypes, to inform prognosis, inform a treatment decision, or identify the rise of a treatment-resistant different subtype.

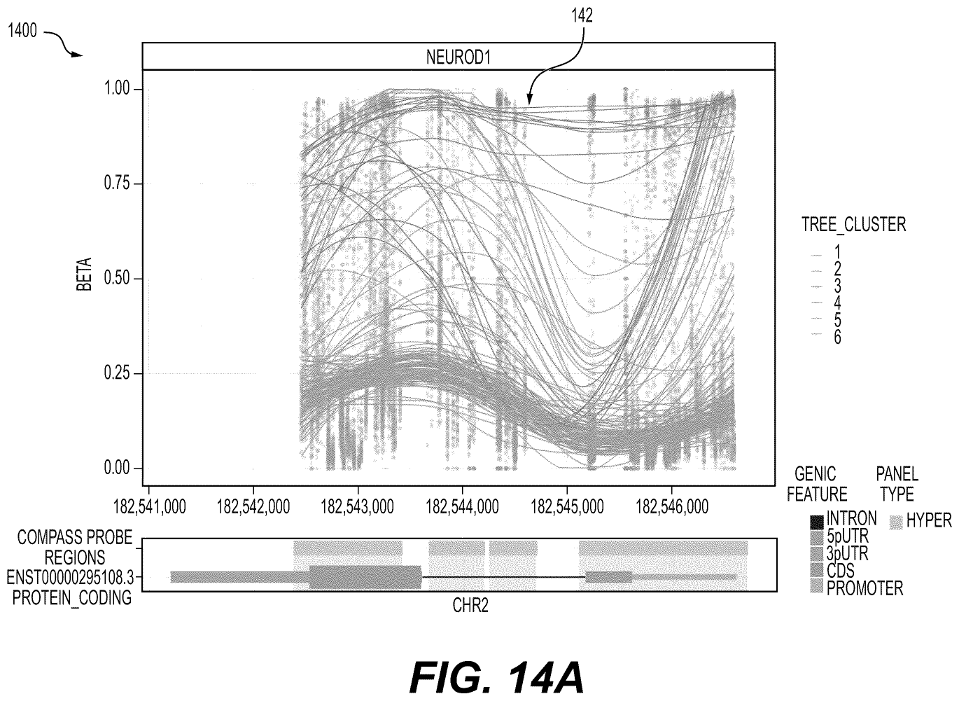

Referring now to FIGS. 13A-13F, dimensionality reduction was performed to reduce the number of variables in the data. The charts shown in FIGS. 13A-13F illustrate how the samples from FIG. 12 were clustering after dimensionality reduction was performed. Principal component analysis (PCA) dimensionality reduction was performed on the data in FIGS. 13A-13F, and Uniform Manifold Approximation and Projection (UMAP) non-linear dimensionality reduction was additionally performed on the data in FIGS. 13C-13D. The colors for participant samples in FIGS. 13A, 13B, and 13E correspond to the tree cluster colors assigned below the dendrogram at the top of FIG. 12 to enable visualization of where the participant samples shown in FIG. 12 map on the two-dimensional charts of FIGS. 13A, 13B, 13E, and 13F. In FIG. 12, there were roughly 6 tree clusters identified based on the methylation signatures of the samples. These 6 tree clusters represent subtypes imposed on the SCLC data based on the methylation signatures of each sample. Accordingly, FIGS. 13A-13F may indicate whether the subtypes inferred using methylation signatures correspond with meaningful differences in the participant samples, or with other clinical or patient information like sex or smoking status, which would not indicate a cancer subtype.

FIG. 13A shows that the participant samples from the tree cluster row in FIG. 12 appear to group in similar clusters in the two-dimensional space defined by principal component 1 (PC1, with 73.1% of the variance explained) and principal component 2 (PC2, with 6.36% of variance explained). FIGS. 13C and 13D show the data set stratified by sex and smoking status, respectively. Given that data points for each participant sample appear evenly distributed throughout the clusters for both sex and smoking status, FIGS. 13C and 13D may indicate that the clusters depicted in the charts do not appear to be attributed to these other covariates that may induce spurious signal when assessing cancer data. Accordingly, the SCLC subtypes identified using methylation status may be attributed to meaningful differences in methylation signatures across the samples, as opposed to confounding variables. FIG. 13E again shows that samples from the same tree cluster—or subtype—tend to cluster together. FIG. 13F generally shows the samples clustering in accordance with cancer status, although there are some outliers. The “stage” row at the top of FIG. 12 indicates the stage of lung cancer, with the blue samples showing non-cancer samples, and the increasingly darker purple samples showing increasingly more advanced stages of cancer. These sample colors in FIG. 12 correspond to the data points shown in FIG. 13F. The cancer samples found clustering with the non-cancer samples in FIG. 13F may have low tumor fraction, and thus it may not be possible to see significant signal in these samples, causing them to cluster with non-cancer samples as opposed to cancer samples.

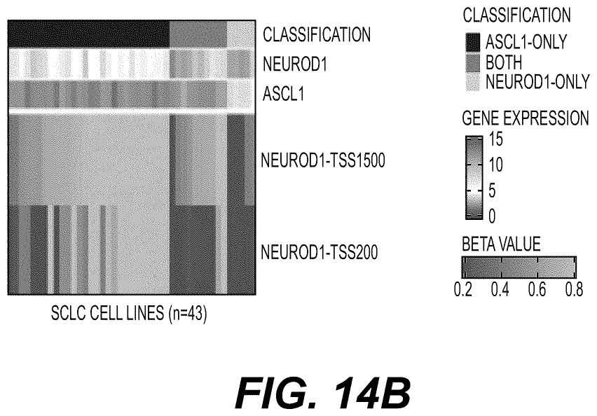

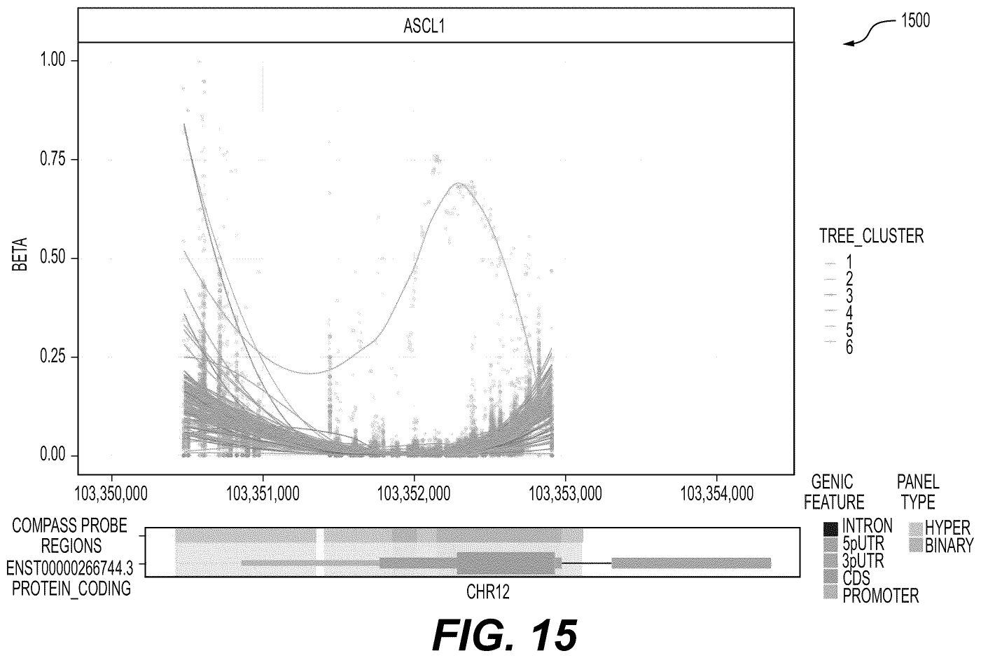

Referring now to FIG. 14A, a line graph 1400 is provided based on the heat map 1200 data illustrated in FIG. 12. Such a line graph 1400 may provide a clearer visual indication of the genomic context of methylation states for each cluster of samples. In this graph, the data presented from FIG. 12 has been restricted to one particular gene, i.e., NEUROD1. The beta values are plotted on the y-axis of the line graph 1400, and the genomic locations are plotted on the x-axis of the line graph 1400. As a general rule of thumb, higher beta values indicate more methylation on DNA fragments in this region. Accordingly, as a representative example of information that may be gleaned from such a graph, focus may be directed to sample cluster 4, which corresponds to the cluster of samples 122 previously discussed in FIG. 12. It can be seen in the line graph 142 that samples associated with sample cluster 4 are all hypermethylated in NEUROD1, which is consistent with what is presented in FIG. 12. It can also be seen that NEUROD1 has hypermethylation both away from the transcription start site (“TSS”) and close to the TSS.

Referring now to FIG. 14B, a heatmap that was recorded in the literature previously is presented. See https://pubmed.ncbi.nlm.nih.gov/33482121/. This heatmap also shows beta values, with more yellow regions indicating greater methylation, and more blue regions indicating less methylation. This heatmap was derived from cell lines and shows that the subtypes defined by expression values of the different transcription factors are associated with methylation values in cell lines at different distances from the TSS of the gene NEUROD1. This heatmap data shows methylation beta values for expression-defined SCLC subtypes called ‘ASCU-only’, ‘NEUROD1-only’, and ‘both’, defined by whether one or both of the genes ASCL1 or NEUROD1 are expressed; this subtype is indicated by the dark band at the top of the heatmap of FIG. 14B. ASCL1-only samples in the heatmap tend to have hypermethylation both near the TSS at about 200 base pairs away, as well as hypermethylation further away from the TSS at about 1,500 base pairs away. This pattern of hypermethylation relative to the TSS of NEUROD1 in the ASCL1-only samples in heatmap 14B is also seen in the samples from cluster 4 as shown in FIG. 14A, which show hypermethylation both at both 200 bp from the NEUROD1 TSS, and at 1500 bp from the NEUROD1 TSS. This correspondence in data may suggest that the subtype identified in sample cluster 4 may correspond to the ASCL1 high subtype. This correlation may thus indicate that it is possible to identify cancer subtypes using methylation signatures as described herein. FIG. 14A also shows that there may be additional layers of stratification that can be learned by looking at methylation patterns in the gene body of NEUROD1, e.g. the samples from cluster 3 in red exhibit mid-range beta values further than 5000 bp from the NEUROD1 TSS, and this is not discernible from FIG. 14B. Accordingly, using a targeted methylation assay, it may be possible to analyze methylation signatures as described herein to identify subtypes previously defined in the literature using conventional methods and to reproduce these patterns using methylation signatures. Embodiments of the disclosure may utilize a targeted methylation assay and pattern recognition to reproduce existing subtypes or discover new subtypes that may be meaningful in, e.g., identifying cancer, determining prognoses, or informing treatment options.

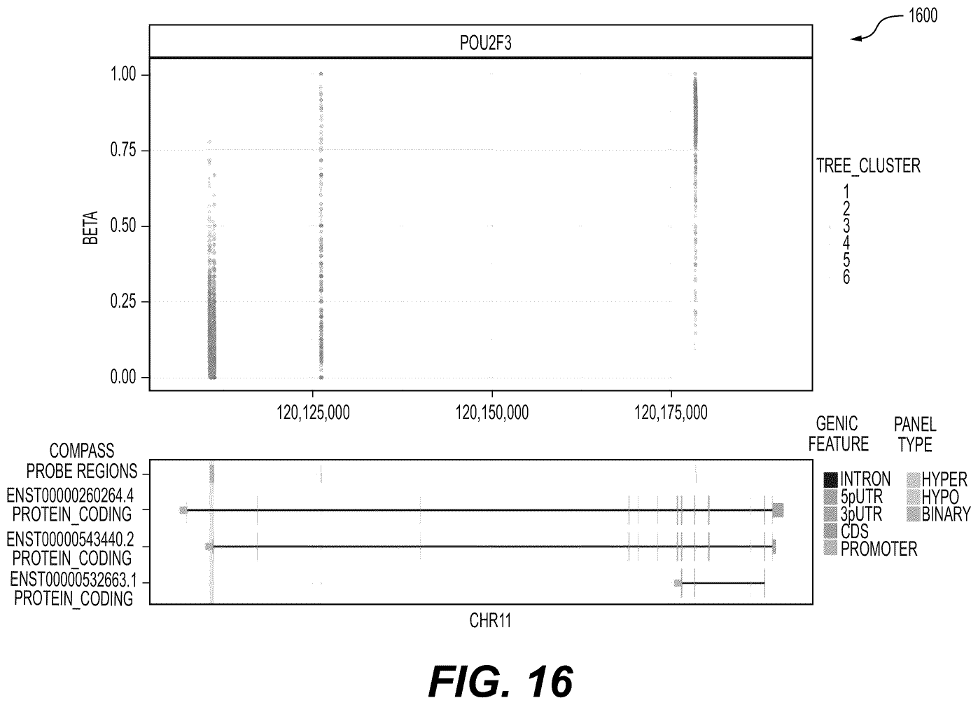

Similar patterns are shown in the line graphs of FIGS. 15-18, which each look at different regions of the genome. Referring now to FIG. 15, a line graph 1500 for methylation values within the gene body of transcription factor ASCL1 is provided based on the data from the heatmap illustrated in FIG. 12. Tree cluster 1 shown in FIG. 15 is separated out from the remainder of the samples and corresponds with the sample 124 shown in FIG. 12.

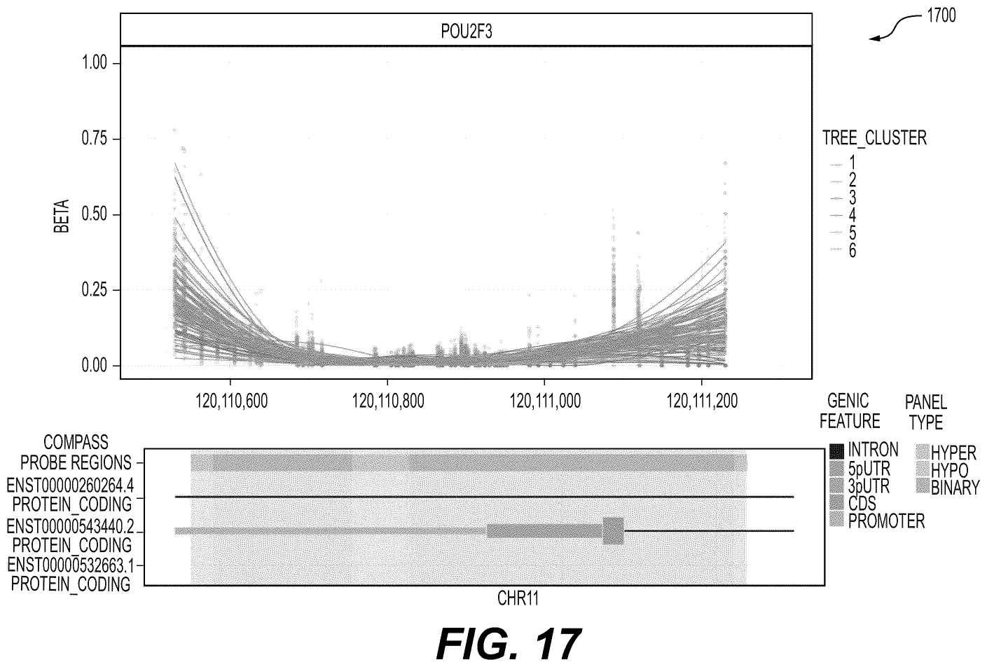

Referring now to FIGS. 16-18, line graphs for methylation values within the gene body of POU2F3 (i.e., 1600-1800) are provided based on the heat map data illustrated in FIG. 12. As described above in reference to FIGS. 14A and 15, FIGS. 16-18 call out the methylation patterns seen in the different genomic regions.

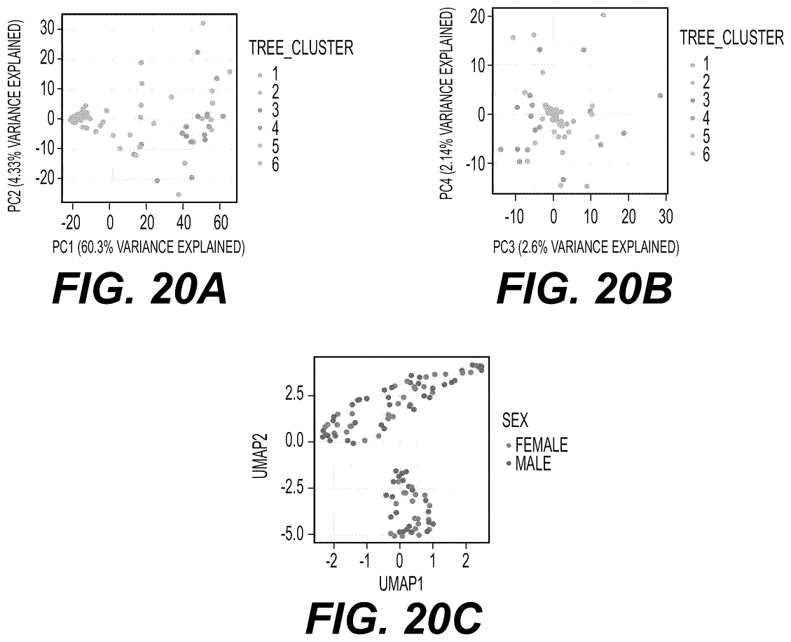

Referring now to FIG. 19, another heatmap 1900 is provided that illustrates methylation beta values. The same kind of analysis as previously described (e.g., with reference to FIG. 12) may be conducted using CpGs within transcription factor motifs or binding sites as the starting point. More particularly, instead of looking at CpGs in the gene bodies of those 4 transcription factors, we can look at CpGs inside of regions that are identified as harboring motifs or binding sites for SCLC-relevant transcription factors, for example motifs of ASCL1, NEUROD1, and POU2F3. For this analysis, motifs were identified from HOCOMOCO [cite], their locations in the genome were identified using PWMTools [cite], and CpGs lying within 50 bp on either side of these motifs (called ‘motif windows’) were identified. Additionally, we filtered CpGs from these motif locations to require that they were significantly differentially methylated when comparing methylation values from SCLC patients to those from non-cancer patients, using a beta-binomial model to identify differential methylation. We also removed CpGs that were present in more than one motif window or that overlapped a CTCF motif window. Upon examination of FIG. 19, structure may also be seen in the heatmap, and that structure is different from the structure observed from the heatmap illustrated in FIG. 12. Stated differently, the clusters learned from this heatmap may be different and may provide different information from those learned in FIG. 12, since an alternate set of genomic regions has been used.

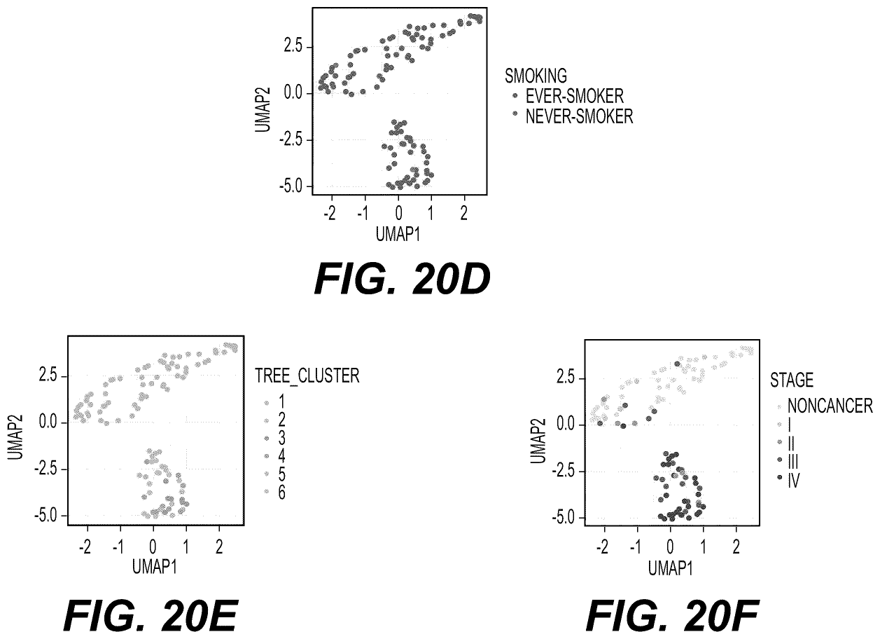

Referring now to FIGS. 20A-F, a plurality of charts are provided that illustrate how the samples from FIG. 19 were clustering, similar to the cluster charts shown in FIGS. 13A-F.

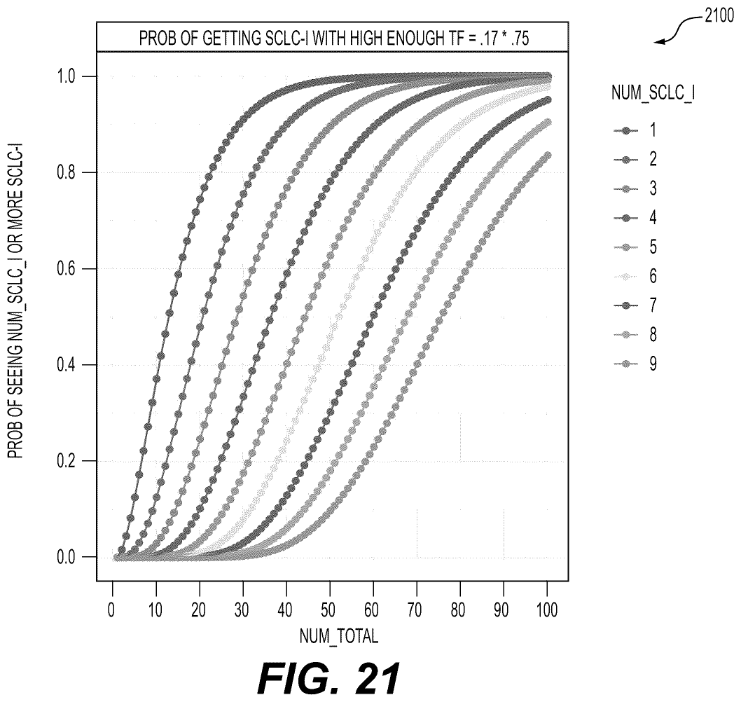

Referring now to FIG. 21, a graph 2100 of sample size considerations are provided. In an embodiment, twice as many samples in plasma should be budgeted as would be needed to build a classifier from tissue data (fewer may suffice in SCLC).

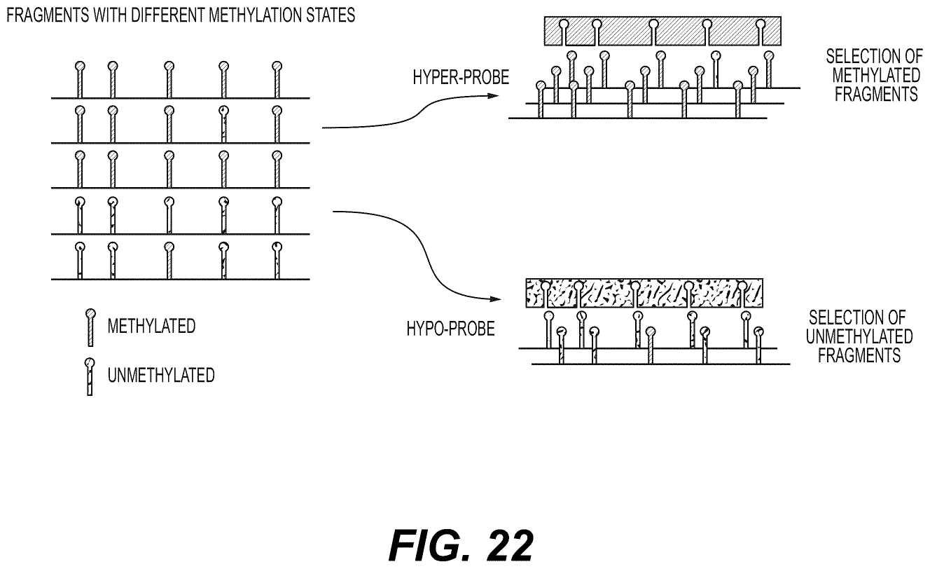

Referring now to FIG. 22, illustrations are presented that indicate how targeted methylation probes may improve test efficiency. More particularly, cfDNA fragments may have different methylation states, e.g., a methylated state and an unmethylated state. Two types of probes, a hyper-probe and a hypo-probe, may be designed to target cfDNA fragments that have the same methylation status at CpGs. However, this does not imply that every captured cfDNA fragment is fully methylated or unmethylated. In an embodiment, each type of probe may select for one type of methylation state (after bisulfite conversion). For instance, the hyper-probe may select for methylated fragments whereas the hypo-probe may select for unmethylated fragments.

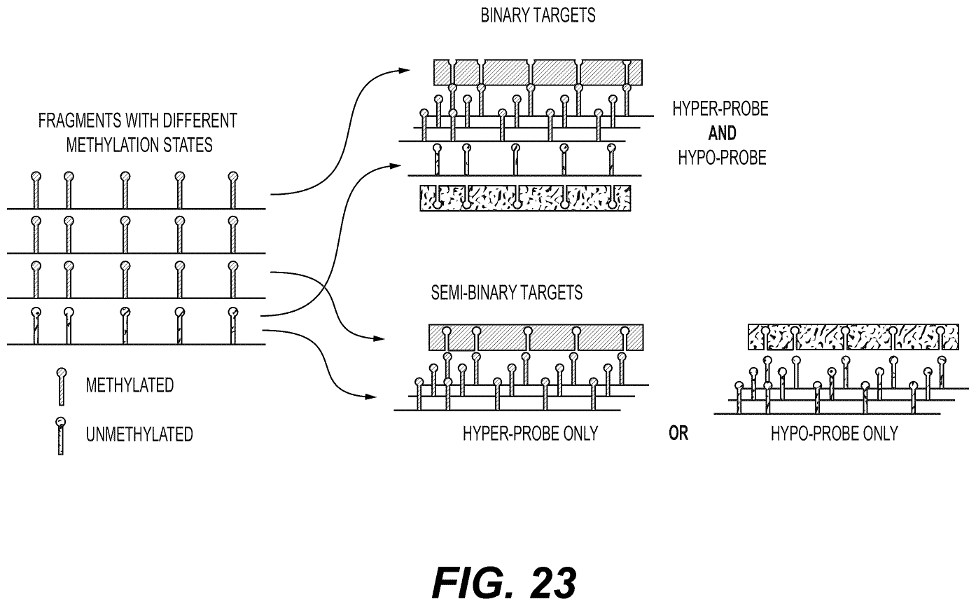

Referring now to FIG. 23, additional illustrations are presented that indicate how targeted methylation probes may improve test efficiency. More particularly, as illustrated in FIG. 22, the cfDNA fragments may have different methylation states. These fragments may be designated as binary targets or semi-binary targets. With respect to the former, binary targets may be targeted by both of the aforementioned types of probes (i.e., hyper-probes and hypo-probes). Conversely, with respect to the latter, semi-binary targets may be targeted by only one kind of probe (e.g., hyper-probe for methylated fragments and hypo-probe for unmethylated fragments).

As discussed above, some embodiments of the disclosure may relate to the detection of resistance mechanisms developed by some cancers under treatment. In this instance, a methylation assay may be used to identify transdifferentiation. To do so, machine learning techniques may be employed to recognize and differentiate between cancer types, e.g., adenocarcinoma and neuroendocrine carcinoma, by training a model to recognize cancer signal origin (CSO) based on histologic type labeling, rather than on anatomic-site-based labeling.

In general, after cancer is detected in a patient (e.g., via the utilization of one or more cancer detection techniques), an appropriate treatment may be prescribed. Subsequent to treatment initiation, regular testing for the cancer (i.e., in the form of MRD for disease monitoring) may be performed at predetermined intervals, the length of which may be dictated by one or more factors (e.g., the severity of the cancer, the type or strength of the treatment, biological characteristics associated with the subject, etc.). Appropriate time intervals may be on the order of weeks, months, or years, e.g., every 2-6 months (e.g., 2, 3, 4, 5, or 6 months), yearly, every 2 years, etc. If a recurrence of the cancer is detected, one or more imaging scans may be performed (e.g., a bone scan, a whole body scan, etc.) and a new treatment course may be determined based on the results of the scans. Additionally or alternatively, the identification that transdifferentiation has occurred may provide an additional indicative metric that the prescribed first-line or current treatment has failed. In this way, a targeted methylation assay may be used for disease monitoring. Further, if transdifferentiation has been detected and a second-line treatment or further therapy is initiated, a targeted methylation assay may continue to be used at regular intervals to determine whether disease burden changes over time. Detecting changes in disease burden may assist in determining whether the second-line therapy is working and/or if or when the second-line therapy fails.

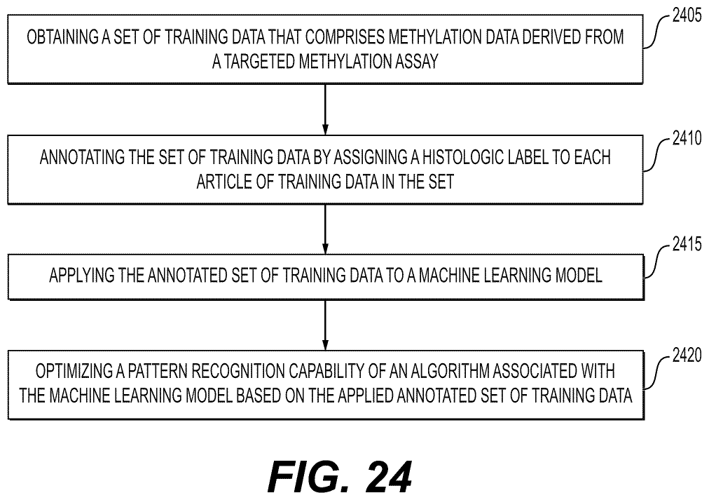

Referring now to FIG. 24, a machine learning model may be trained to determine whether MRD is present within a biological sample and, if it is, whether the cancer cells associated with the MRD have developed resistance to a treatment.

At step 2405, a set of training data may be obtained from a source. In an embodiment, the source may be an accessible database, and the training data may comprise DNA methylation data that was derived from a targeted methylation sequencing assay performed on a biological sample acquired from a training subject. In an embodiment, the accessible database may be continuously or periodically updated with new training data.

At step 2410, the set of training data may be annotated so that each article of training data in the set is assigned a histologic label, as opposed to an anatomic-site-based label. The histologic type of a cancer is routinely determined in clinical practice by pathology review of a tissue or other specimen. Training data may be labeled to indicate whether DNA methylation data was obtained from a source with a given type of cancer, e.g., lung cancer, prostate cancer, etc. Labels may also include the age, sex, race, or other information pertaining to an individual source of DNA methylation data (e.g., the responsiveness of a patient to a particular course of treatment, etc.).

At step 2415, the annotated training data may be applied to the machine learning model. In an embodiment, the machine learning model may be virtually any type of supervised, unsupervised, or mixed machine learning model chosen by a user.

At step 2420, the application of the annotated set of training data to the machine learning model may optimize a pattern recognition capability of an algorithm associated with the machine learning model. More particularly, the algorithm of the machine learning model may be trained to: A) identify whether MRD exists in a test subject (i.e., by determining whether any cancer signal exists in the biological sample or by using annotation information if residual disease has been detected by imaging or other clinical tests); and, if so B) determine whether the cancer cells associated with the MRD have developed a resistance mechanism to a previously administered or on-going treatment. In this regard, the algorithm may be trained to recognize whether transdifferentiation has occurred by identifying that at least some of the cancer cells have transformed from a first cancer type (e.g., adenocarcinoma) to the second cancer type (e.g., small cell neuroendocrine carcinoma) based on the histologic labels. In an optional embodiment, the machine learning model may be further trained to output a new treatment recommendation responsive to identifying that a resistance mechanism has been developed and/or identifying the type of resistance mechanism. In an embodiment, the new treatment recommendation may include a suggestion of a new drug therapy to administer to a patient, a surgical procedure to conduct on the patient, an adjustment to a liquid biopsy collection (e.g., blood draw) schedule for the patient, etc.

In an embodiment, the new treatment recommendation may consider how well a test subject may respond to various types of potential treatments. More particularly, the machine learning model may be able to identify a biological pattern in a test subject's blood draw and anticipate (e.g., by leveraging data procured through one or more dedicated training phases that identify how well other test subjects with similar biological patterns responded to different types of treatment) how well the test subject may respond to the potential treatment types. In an embodiment, the new treatment recommendation may contain a recommendation for a disease monitoring schedule based upon a determination of how well the subject may respond to a potential treatment (e.g., 6 month detection interval proposed if test subject is anticipated to respond well to the treatment versus 2 month detection interval proposed if the test subject is anticipated to respond less well to the treatment, etc.).

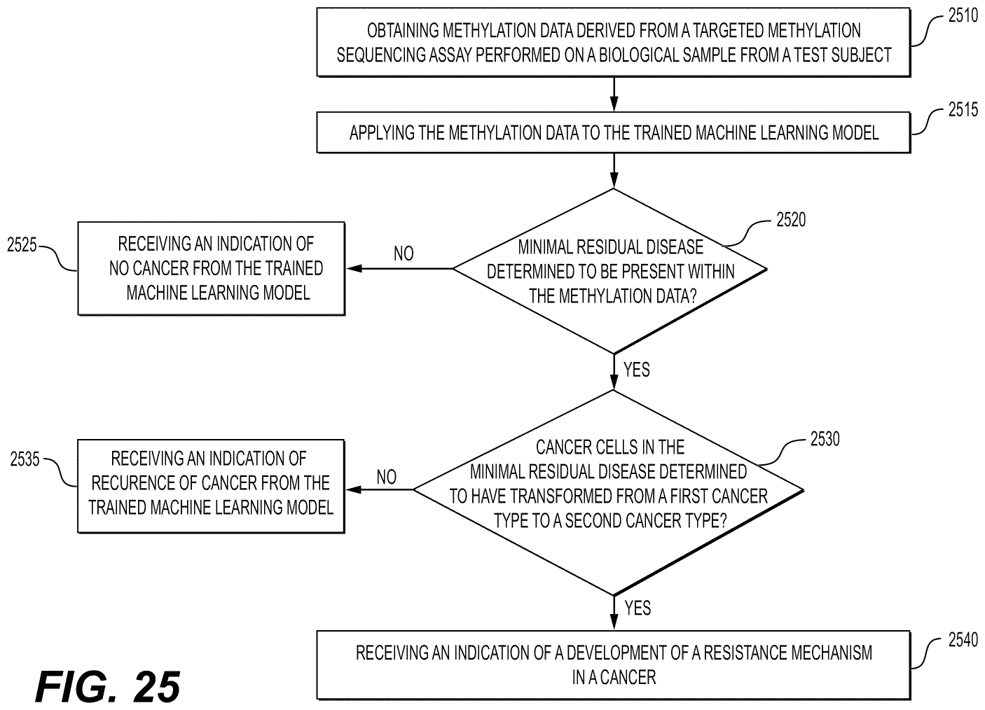

Referring now to FIG. 25, a trained machine learning model may be utilized to determine whether MRD is present and, if it is, whether the cancer cells have developed resistance to an ongoing treatment.

At step 2510, methylation data derived from a targeted methylation sequencing assay performed on a biological sample acquired from a test subject may be obtained. At step 2515, the methylation data may be applied to a trained machine learning model (e.g., the machine learning model discussed above in reference to FIG. 24). The methylation data may be processed by an algorithm of the trained machine learning model and an output result may be received. More particularly, the machine learning model may determine, at step 2520, whether MRD is present within the test subject. Such a determination may be facilitated by identifying whether any cancer signal is present within the methylation data. Responsive to determining, at step 2520, that no cancer signal is present within the methylation data, the machine learning model may, at step 2525, output a result indicating that no cancer has been detected. Alternatively, responsive to determining, at step 2520, that MRD is present, the machine learning model may, at step 2530, further determine whether a resistance mechanism has been developed by the cancer cells in response to an on-going treatment. This determination may be facilitated by identifying whether at least a portion of the detected cancer cells have transformed from a first cancer type (i.e., the cancer type that was initially identified and that a current treatment was originally prescribed for) to another cancer type. The algorithm of the trained machine learning model may be able to execute this identification based on the training data sets annotated with histologic labels for each cancer type. More particularly, a robust separation between adenocarcinomas and neuroendocrine carcinomas may be identified when predicting the histologic type from detected ctDNA in a liquid biopsy, e.g., blood plasma, sample. Responsive to determining, at step 2530, that the cancer cells associated with the MRD are equivalent to the first cancer type, the machine learning model may, at step 2535, output a result indicating that the first cancer type has recurred in the test subject. Alternatively, responsive to determining, at step 2530, that at least a portion of the cancer cells associated with the MRD have transformed from a first cancer type to a second cancer type, the machine learning model may, at step 2540, output a result indicating that the cancer cells of the first cancer type have developed a resistance to an on-going treatment. In an optional embodiment, the trained machine learning model may further output a treatment recommendation based on the determination that the cancer has developed a resistance to the treatment. For instance, the treatment recommendation may suggest an alternative therapy that may be administered to the test subject, a potential surgery that may be conducted on the test subject, an adjustment to a liquid biopsy collection (e.g., blood draw) schedule for the test subject, etc.

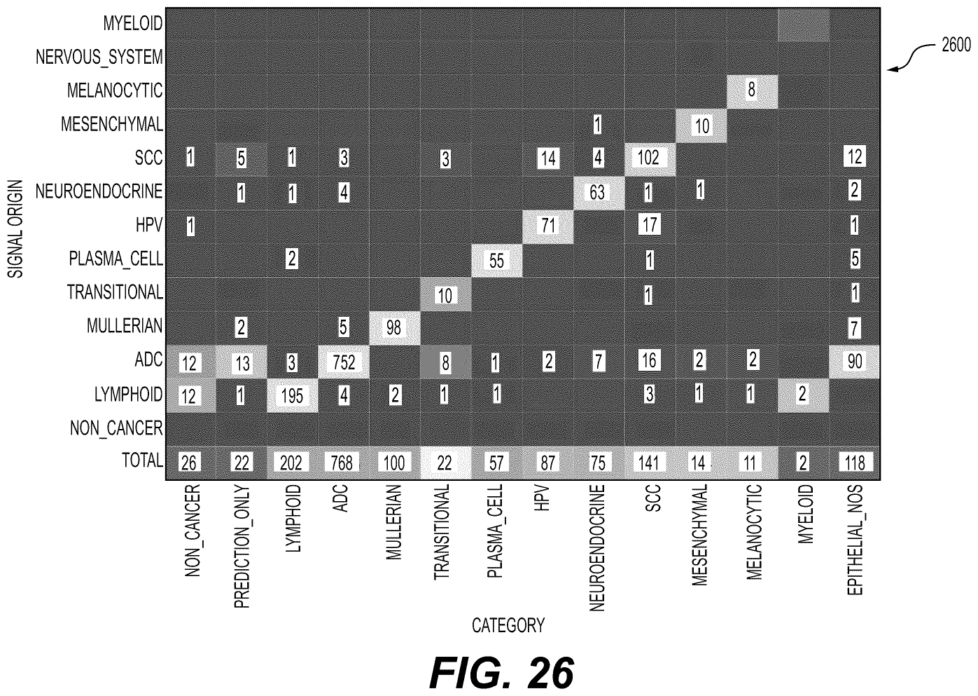

Referring now to FIG. 26, a confusion matrix 2600 is presented that is resultant from an exemplary application of methylation data derived from a set of test subjects to a trained machine learning model. In an embodiment, the machine learning model was trained by utilization of histologic cancer types as CSO labels. For clarity, only samples with detected cancer signal are shown. Column labels correspond to the ground truth class of the histologic type, and row labels indicate the classifier result. For interpretation purposes, “adc” corresponds to adenocarcinoma; “hpv” corresponds to HPV-positive squamous cell carcinoma; “scc” corresponds to HPV-negative squamous cell carcinoma, and “epithelial_nos” corresponds to epithelial cancer with histologic type not otherwise specified. An examination of the confusion matrix may reveal the accuracy of the prediction results of the machine learning model. The confusion matrix indicates how often the classifier produces a call from an individual's blood draw that matches the diagnosis determined for that individual based on the clinical workup. Experimental results indicate that a targeted methylation sequencing assay may be used for disease monitoring and may be used in parallel for disease burden quantification or MRD detection and a detection of transdifferentiation of the cancer.

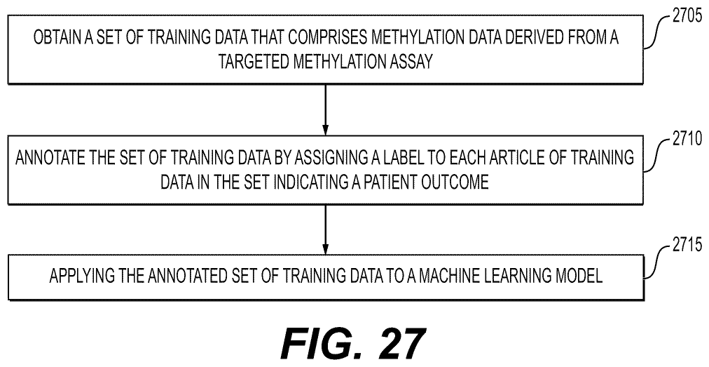

Referring now to FIG. 27, a machine learning model may be trained to generate a subject prognostic score. At step 2705, a set of training data may be obtained from a source. In an embodiment, the source may be an accessible database, and the training data may comprise DNA methylation data that was derived from a targeted methylation sequencing assay performed on a biological sample, e.g., a liquid sample such as a blood sample, acquired from one or more training subjects. In an embodiment, the accessible database may be continuously or periodically updated with new training data. In an embodiment, the subject methylation data on which the final prognosis score is based may be the same methylation data that is utilized in other cancer classification determinations. More particularly, both prognostic and cancer classification determinations may be made from the same blood sample, which may prevent a subject from having to experience an additional liquid sample collection such as another blood draw.

Aspects of the embodiments may employ filtering of cfDNA fragments based on their methylation status. For example, embodiments of the disclosure may identify regions in the genome where the presence of some unusually methylated fragments are capable of separating cancer subjects with good versus poor prognosis, or separating cancer subjects from non-cancer subjects. In an embodiment, the DNA methylation data utilized to train the machine learning model may only contain those fragments of cfDNA that are abnormally methylated. More particularly, a DNA fragment may be included in subsequent training and scoring when its methylation pattern is unlikely to be observed among the cfDNA fragments seen in healthy non-cancer study participants.

Additionally to the foregoing, the training data may be based on a methylation feature set that is substantially the same as that used in the aforementioned Galleri classifier (i.e., the feature set may be representative of those abnormally methylated CpG sites that were identified for cancer detection or for differentiating different cancer signal origins). For example, a P_prognostic_CSO labeling approach and a P_prognostic_Galleri_fragment labeling approach may both utilize methylation patterns that were typically used to distinguish different cancer types. More particularly, each of the foregoing approaches utilizes the same set of methylation sites that were used to train the Galleri classifier (i.e., those CpG sites in the genome relied on for cancer detection and/or cancer signal origin). Alternatively, the training data may be based on a different feature set that corresponds to methylation sites that are deemed particularly informative for subject prognosis. For example, a P_prognostic_fragment approach may be a from-scratch feature identification in which a methylation panel is examined for unique methylation sites that may be of particular relevance to a subject prognosis (i.e., that are more correlated to the subject's prognosis). There may or may not be overlap between the traditional Galleri methylation patterns and the methylation patterns identified as of particular relevance to determining subject prognosis.

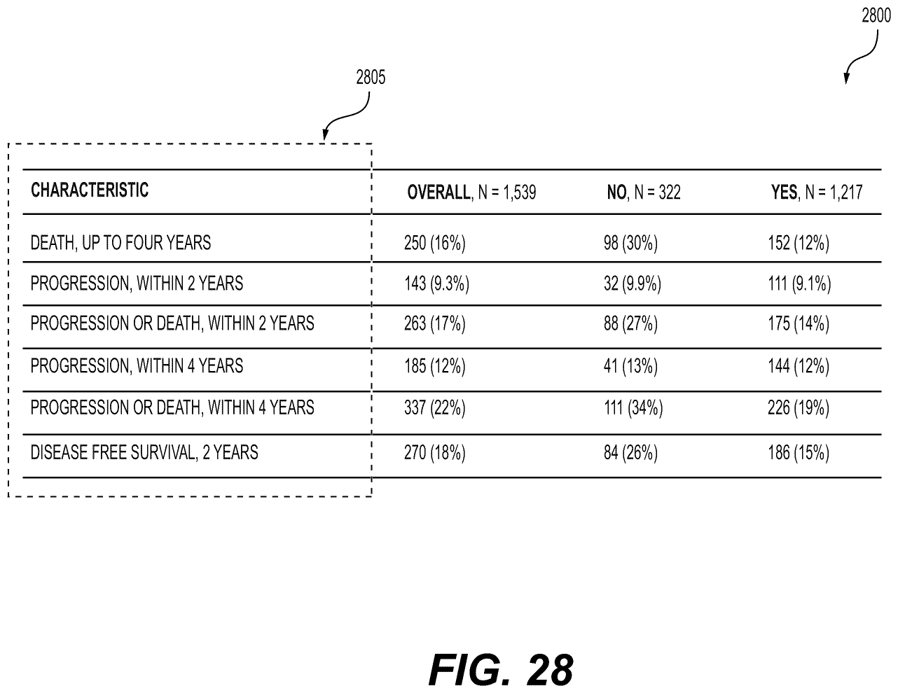

At step 2710, the set of training data may be annotated so that each article of training data in the set (i.e., each set of DNA methylation data associated with a training subject) is assigned a label indicating an outcome that the disease had for the training subject. In an embodiment, possible labels may include: subject death after X years, disease progression in the subject within X years, disease progression and subject death within X years, subject metastasis or reoccurrence after X years, and subject is disease free and surviving after X years. For instance, referring to FIG. 28, a plurality of sample labels utilized on a set of training data are provided. The training labels in the set 2805 provide an indication of whether a training subject was alive or dead after a predetermined number of years and/or whether the cancer is gone, stagnant, or has progressed (e.g., even after surgery or after treatment it continued to grow or started growing again).

Referring back to FIG. 27, at step 2715, the annotated training data may be applied to the machine learning model. In an embodiment, the machine learning model may be virtually any type of supervised, unsupervised, or mixed machine learning model chosen by a user. In an embodiment, the application of the annotated training data may optimize a pattern recognition capability of an algorithm associated with the machine learning model to learn which patterns of methylation in cancer subjects are indicative of subject prognosis. For instance, the machine learning model may learn that the presence of a group of abnormally methylated CpG sites (Group A) results in a poor subject prognosis (e.g., disease progression and death within 2 years) whereas a different group of abnormally methylated CpG sites (Group B) results in a more favorable subject prognosis (no disease progression and subject survival after 2 years).

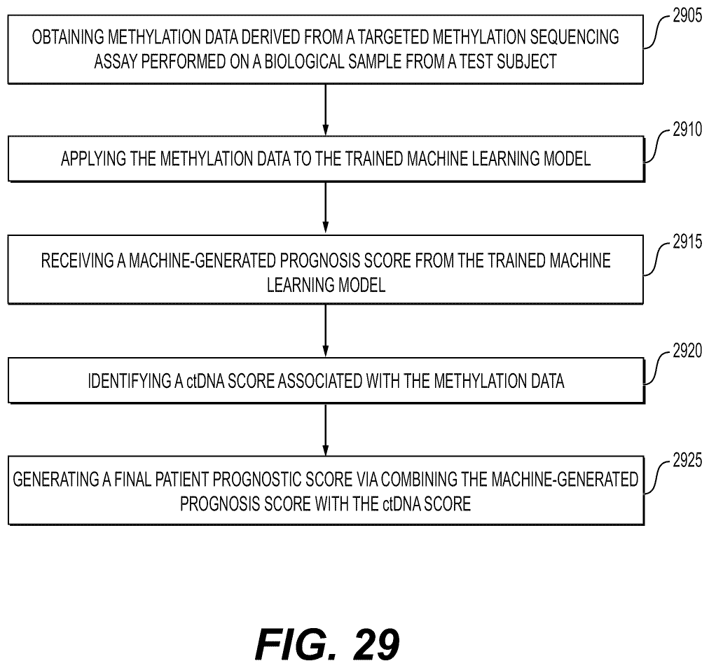

Referring now to FIG. 29, a method of generating a final prognostic score for a patient from the combination of a model-generated prognosis score and a ctDNA score is disclosed.

At step 2905, methylation data derived from a targeted methylation sequencing assay performed on a biological sample acquired from a test subject may be obtained. The biological sample may be a liquid biological sample, such as a blood draw. At step 2910, the methylation data derived from the targeted methylation sequencing assay may be applied to a trained machine learning model (e.g., the machine learning model discussed above in reference to FIG. 24). The methylation data may be processed by an algorithm of the trained machine learning model and an output result in the form of a prognosis score may, at step 2915, be received. In an embodiment, the prognosis score may have a value in the range 0 to 1, with 0 indicating a methylation pattern highly similar to those of patients in the training population with good prognosis and 1 indicating a methylation pattern highly similar to those of patients in the training population with poor prognosis. At step 2920, a ctDNA score may be identified that is representative of the fraction of cfDNA in the biological sample that is derived from tumor rather than non-cancerous tissues (i.e., tumor fraction). In an embodiment, the tumor fraction may be estimated using one or more computational tools and/or methods known in the art. In an embodiment, the ctDNA score may be obtained by taking the log 10 of the computed tumor fraction value, i.e., log 10 (mVAF). At step 2925, a final patient prognostic score may be generated by combining the machine-generated prognosis score received at step 2915 with the ctDNA score identified in step 2920. In an embodiment, the ctDNA score and the machine-generated prognosis score may be combined in one or more different ways. For example, one non-limiting way of combining the ctDNA score and the machine-generated prognosis score may be to multiply the two metrics together. Other ways of combining the ctDNA score and the machine-generated prognosis score include by adding the two metrics together or by combining them through virtually any other mathematical means.

In an embodiment, the final patient prognostic score may be utilized as the basis for one or more downstream actions and/or determinations. For instance, the range of values that the final patient prognostic score falls within may provide an indication of how good or poor the prognosis is. For example, a good prognosis indication may be provided if the final patient prognostic score falls within a first range of values (e.g., 0 to 0.3), a moderate prognosis indication may be provided if the final patient prognostic score falls within a second range of values (e.g., 0.3-0.6), and a poor prognosis indication may be provided if the final patient prognostic score falls within a third range of values (e.g., 0.6-1). These ranges are exemplary only, and different groupings or numbers of groupings may be utilized according to ranges that are clinically relevant. In other examples, a threshold may be set and the final patient prognostic score may be compared to the threshold. A value below the threshold may indicate a good prognosis score, while a value above the threshold may indicate a poor prognosis indication.

Additionally or alternatively, various recommendations (e.g., recommendations for a type of clinical study to participate in, recommendations for a disease management process, recommendations for a follow-up regimen during/after treatment, etc.) may be dynamically provided to the subject, clinician, and/or clinical trial supervisor based on the range of values that the final patient prognostic score falls within or whether the value is above or below the threshold. For example, if the final patient prognostic score falls within the third range of values described above, or above the threshold, then a recommendation may be created that suggests that the subject be enrolled in a more aggressive clinical trial in an attempt to improve the poor projected prognosis. In other aspect, a more aggressive treatment protocol may be recommended, or a more frequent follow-up schedules during/after treatment may be recommended (for example, more frequent testing, scans, biopsy collection (e.g., blood draw) or other follow-up regimens may be recommended). A recommendation may be created that suggests a modification to a subject's biopsy collection (e.g., blood draw) schedule following treatment by which the blood draws may occur more frequently. In still other aspects, if a patient identified as having a poorer prognosis is enrolled in a clinical trial, then modifications may be made to the clinical trial enrollment or resulting data analysis so that the prognosis is taken into account as a relevant variable to potentially control for.

As another example, if the final patient prognostic score falls within the first range of values described above, or below the threshold, then a less aggressive treatment protocol may be recommended, or a less frequent follow-up regimen during/after treatment may be recommended. For example, more frequent testing, scans, biopsy collection (e.g., blood draw) or other follow-up regimens may be recommended). A recommendation may be created that suggests a modification to a subject's biopsy collection (e.g., blood draw) schedule following treatment by which the blood draws may occur less frequently. In still other aspects, if a patient identified as having a better prognosis is enrolled in a clinical trial, then modifications may be made to the clinical trial enrollment or resulting data analysis so that the prognosis is taken into account as a relevant variable to potentially control for.

Further to the foregoing, the final patient prognosis score may be leveraged in the clinical trial context in various ways. More particularly, knowledge of a patient's prognosis may be valuable in the context of clinical trial design for patient stratification and inclusion criteria in order to increase event rate and power. For example, given a particular clinical trial, a patient population may be divided into distinct subgroups based on prognosis indications derived from the final patient prognosis score.

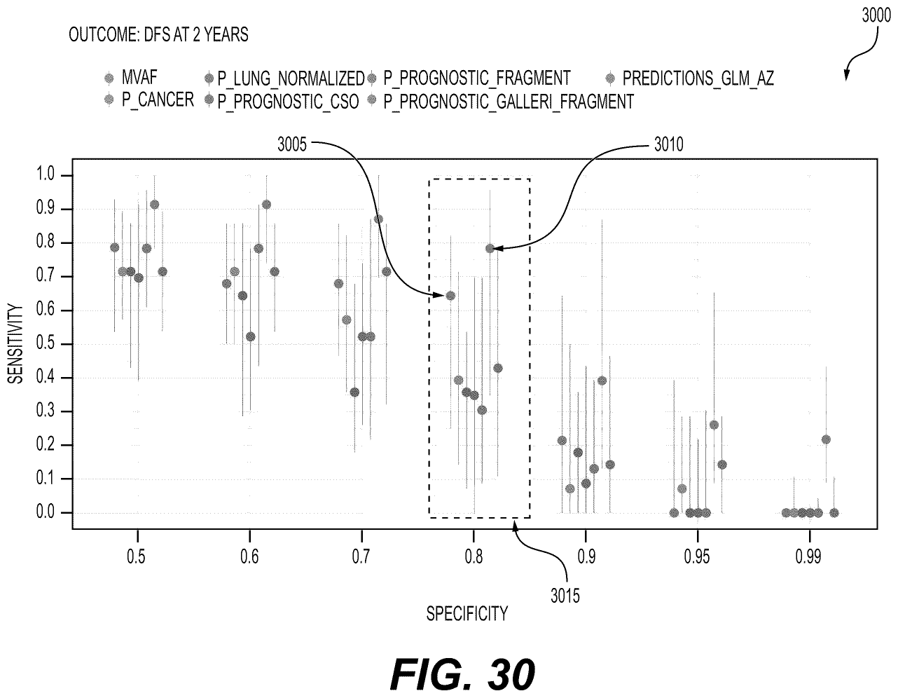

Referring now to FIG. 30, a graph 3000 is presented that illustrates a plurality of patient prognosis predictions for one cancer type (i.e., squamous cell carcinoma of the lung). In the graph 3000, multiple sets of prognostic predictions are presented at different levels of specificity and each set contains prognostic predictions generated using different approaches. For the purposes of this discussion, two specific prognostic prediction metrics (3005, 3010) in one set of prognostic predictions 3015 in the graph 3000 may be examined. More particularly, the relevant set 3015 may contain an indication of a first prognostic prediction 3005 corresponding to just the tumor fraction value (i.e., mVAF) and another prognostic prediction 3010 (i.e., P_prognostic_Galleri_fragment) corresponding to the combination of the machine-generated prognosis score with the ctDNA score, as previously disclosed above. Examination of the graph 3000 indicates that the prognostic prediction 3010 associated with the P_prognostic_Galleri_fragment generally has greater specificity and sensitivity than the tumor fraction value 3005, which is an indication that the former provides better patient prognosis than just the mVAF value alone. Additional observations that may be made from the graph 3000 are that the prognostic prediction 3010 associated with the P_prognostic_Galleri_fragment has similar specificity in each set as the P_prognostic_CSO metric and the P_prognostic_fragment but outperforms both of the foregoing in sensitivity.

In some embodiments, the methods, systems, and/or classifier(s) of the present disclosure can be used to detect the presence (or absence) of cancer, monitor cancer progression or recurrence, monitor therapeutic response or effectiveness, monitor transdifferentiation of a cancer, determine a presence or monitor minimum residual disease (MRD), quantify disease burden, generate a patient prognosis score, or any combination thereof. In some embodiments, the systems and/or classifier may be used to identify histologic types or molecular subtypes of cancer. For instance, the systems and/or classifiers may be used to identify a cancer as of any of the following cancer types: epithelial, nervous system, lymphoid, myeloid, plasma cells, mesenchymal, melanocytic, neuroendocrine, and germ cells, and to further distinguish for all epithelial cancers into histologic types, namely adenocarcinoma (adc), HPV-associated squamous cell carcinoma (hpv), non-HPV-associated squamous cell carcinoma (scc), carcinoma of Mullerian origin (mullerian), and transitional cell carcinoma. In some embodiments, subtypes for known cancer types can be identified, for example type I epithelial ovarian cancer and type II high-grade serous ovarian cancer or different molecular subtypes of SCLC that can be identified by dominant transcription factors. In some embodiments, a test report can be generated to provide a patient with their test results, including, for example, a probability score that the patient has a disease state (e.g., cancer), a type of disease (e.g., a type of cancer), and/or a histologic type or molecular subtype (i.e., when generating the report, a CSO label can be generated for specific people based on co-factors such as sex, smoker status, etc.). In some embodiments, the methods and/or classifier of the present disclosure are used to identify a cancer type, histologic type, or molecular subtype in a suspect that has been diagnosed with a cancer. In some embodiments, this identification of a cancer type, histologic type, or molecular subtype can use the same blood draw, plasma sample and sequencing results that have been used to detect cancer in a subject participating in a cancer early detection or screening program. According to aspects of the disclosure, the methods and systems of the present disclosure can be trained to detect or classify multiple cancer indications. For example, the methods, systems, and classifiers of the present disclosure can be used to detect the presence of one or more, two or more, three or more, five or more, or ten or more different types of cancer, histologic types, or molecular subtypes. In some embodiments, the cancer is one or more of adenocarcinoma, HPV-associated squamous cell carcinoma, non-HPV-associated squamous cell carcinoma, a type I carcinoma of Mullerian origin, a type II cancer of Mullerian origin, transitional cell carcinoma, or a neuroendocrine carcinoma like small cell carcinoma with a molecular subtype identified by different transcription factors.

A dataset may be generated for sequence data obtained from a cancer patient over any desired set of time points and analyzed in accordance with the methods of the disclosure to monitor a cancer state in the patient. In some embodiments, a first time point is before a cancer treatment (e.g., before a resection surgery or a therapeutic intervention), and the second time point is after a cancer treatment (e.g., after a resection surgery or therapeutic intervention), and the method is utilized to monitor the effectiveness of the treatment. In other embodiments, both the first and second time points are before a cancer treatment (e.g., before a resection surgery or a therapeutic intervention). In still other embodiments, both the first and the second time points are after a cancer treatment (e.g., before a resection surgery or a therapeutic intervention) and the method is used to monitor the effectiveness of the treatment or loss of effectiveness of the treatment. In still other embodiments, the dataset may be generated for sequence data obtained from a cancer patient at a first and second time point and analyzed. e.g., to monitor cancer progression, to determine if a cancer is in remission (e.g., after treatment), to monitor or detect residual disease or recurrence of disease, or to monitor treatment (e.g., therapeutic) efficacy, or if a cancer has changed its histologic type or molecular subtype while developing a resistance to a cancer treatment.

In some embodiments, the first and second time points are separated by an amount of time that ranges from about 15 minutes up to about 30 years, such as about 30 minutes, such as about 1, 2, 3, 4, 5, 6, 7, 8, 9, 10, 11, 12, 13, 14, 15, 16, 17, 18, 19, 20, 21, 22, 23, or about 24 hours, such as about 1, 2, 3, 4, 5, 10, 15, 20, 25 or about 30 days, or such as about 1, 2, 3, 4, 5, 6, 7, 8, 9, 10, 11, or 12 months, or such as about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5 or about 30 years. In other embodiments, datasets can be generated for sequence data obtained from the patient at least once every 3 months, at least once every 6 months, at least once a year, at least once every 2 years, at least once every 3 years, at least once every 4 years, or at least once every 5 years.

In still another embodiment, information obtained from any method described herein can be used to make or influence a clinical decision (e.g., diagnosis of cancer, treatment selection, assessment of treatment effectiveness, etc.). For example, in one embodiment, a physician can prescribe an appropriate treatment (e.g., a resection surgery, radiation therapy, chemotherapy, and/or immunotherapy) based on the utilization of the dataset in a classification process. In some embodiments, information such as classification based on the dataset can be provided as a readout to a physician or subject. In some embodiments, classification based on the dataset can indicate the effectiveness of a cancer treatment.

In some embodiments, the treatment is one or more cancer therapeutic agents selected from the group including a chemotherapy agent, a targeted cancer therapy agent, a differentiating therapy agent, a hormone therapy agent, and an immunotherapy agent. For example, the treatment can be one or more chemotherapy agents selected from the group including alkylating agents, antimetabolites, anthracyclines, anti-tumor antibiotics, cytoskeletal disruptors (ta×ans), topoisomerase inhibitors, mitotic inhibitors, corticosteroids, kinase inhibitors, nucleotide analogs, platinum-based agents and any combination thereof. In some embodiments, the treatment is one or more targeted cancer therapy agents selected from the group including signal transduction inhibitors (e.g., tyrosine kinase and growth factor receptor inhibitors), histone deacetylase (HDAC) inhibitors, retinoic receptor agonists, proteosome inhibitors, angiogenesis inhibitors, and monoclonal antibody conjugates. In some embodiments, the treatment is one or more differentiating therapy agents including retinoids, such as tretinoin, alitretinoin and bexarotene. In some embodiments, the treatment is one or more hormone therapy agents selected from the group including anti-estrogens, aromatase inhibitors, progestins, estrogens, anti-androgens, and GnRH agonists or analogs. In one embodiment, the treatment is one or more immunotherapy agents selected from the group comprising monoclonal antibody therapies such as rituximab (RITUXAN) and alemtuzumab (CAMPATH), non-specific immunotherapies and adjuvants, such as BCG, interleukin-2 (IL-2), and interferon-alfa, immunomodulating drugs, for instance, thalidomide and lenalidomide (REVLIMID). The appropriate cancer therapeutic agent can be selected based on characteristics such as the type of tumor, histologic type, molecular subtype, cancer stage, previous exposure to cancer treatment or therapeutic agent, and other characteristics of the cancer.

In addition to a standard desktop, or server, it is fully within the scope of this disclosure that any computer system capable of the required storage and processing demands would be suitable for practicing the embodiments of the present disclosure. This may include tablet devices, smart phones, pin pad devices, and any other computer devices, whether mobile or even distributed on a network (i.e., cloud based).

Unless specifically stated otherwise, as apparent from the following discussions, it is appreciated that throughout the specification discussions utilizing terms such as “processing,” “computing,” “calculating,” “determining”, analyzing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” may refer to any device or portion of a device that processes electronic data, e.g., from registers and/or memory to transform that electronic data into other electronic data that, e.g., may be stored in registers and/or memory. A “computer,” a “computing machine,” a “computing platform,” a “computing device,” or a “server” may include one or more processors.

In accordance with various implementations of the present disclosure, the methods described herein may be implemented by software programs executable by a computer system. Further, in an exemplary, non-limited implementation, implementations may include distributed processing, component/object distributed processing, and parallel payment. Alternatively, virtual computer system processing may be constructed to implement one or more of the methods or functionality as described herein.

Although the present specification describes components and functions that may be implemented in particular implementations with reference to particular standards and protocols, the disclosure is not limited to such standards and protocols. For example, standards for Internet and other packet switched network transmission (e.g., TCP/IP, UDP/IP, HTML, HTTP, etc.) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the steps of methods discussed are performed in one embodiment by an appropriate processor (or processors) of a processing (i.e., computer) system executing instructions (computer-readable code) stored in storage. It will also be understood that the disclosed embodiments are not limited to any particular implementation or programming technique and that the disclosed embodiments may be implemented using any appropriate techniques for implementing the functionality described herein. The disclosed embodiments are not limited to any particular programming language or operating system.

It should be appreciated that in the above description of exemplary embodiments, various features of the embodiments are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that a claimed embodiment requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment.

Furthermore, while some embodiments described herein include some but not other features included in other embodiments, combinations of features of different embodiments are meant to be within the scope of the present disclosure, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the function.

In the description provided herein, numerous specific details are set forth. However, it is understood that embodiments of the present disclosure may be practiced without these specific details. In other instances, well-known methods, structures, and techniques have not been shown in detail in order not to obscure an understanding of this description.

Similarly, it is to be noticed that the term coupled, when used in the claims, should not be interpreted as being limited to direct connections only. The terms “coupled” and “connected,” along with their derivatives, may be used. It should be understood that these terms are not intended as synonyms for each other. Thus, the scope of the expression a device A coupled to a device B should not be limited to devices or systems wherein an output of device A is directly connected to an input of device B. It means that there exists a path between an output of A and an input of B which may be a path including other devices or means. “Coupled” may mean that two or more elements are either in direct physical or electrical contact, or that two or more elements are not in direct contact with each other but yet still co-operate or interact with each other.

Thus, while there has been described what are believed to be the preferred embodiments of the present disclosure, those skilled in the art will recognize that other and further modifications may be made thereto without departing from the spirit of the present disclosure, and it is intended to claim all such changes and modifications as falling within the scope of the present disclosure. For example, any formulas given above are merely representative of procedures that may be used. Functionality may be added or deleted from the block diagrams and operations may be interchanged among functional blocks. Steps may be added or deleted to methods described within the scope of the present disclosure.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations and implementations are possible within the scope of the disclosure. Accordingly, the disclosure is not to be restricted except in light of the attached claims and their equivalents.

What is claimed is:

1. A method of detecting a subtype of a disease state using a system, the method comprising:
receiving, at an input component of the system, a set of sequence reads associated with a nucleic acid sample;
generating, using a processor of the system and via analysis of the set of sequence reads, methylation data; and
analyzing, using the processor, the methylation data to identify the subtype of the disease state.

2. The method of claim 1, further comprising:
applying, using the processor, information associated with the identified subtype as training input to a disease state classifier; and
utilizing the disease state classifier trained on the information associated with the identified subtype on one or more subsequent sets of nucleic acid samples.

3. The method of claim 1, wherein the subtype of the disease state includes an embryologic origin of the cancer cells.

4. The method of claim 1, wherein the subtype of the disease state includes a histologic subtype.

5. The method of claim 1, wherein the subtype of the disease state includes a molecular subtype.

6. The method of claim 5, wherein the molecular subtype has previously been defined based on protein expression identified using a cancer tissue sample.

7. The method of claim 5, wherein the molecular subtype has previously been defined based on gene expression identified using a cancer tissue sample.

8. The method of claim 5, wherein the molecular subtype has previously been defined based on genomic alterations identified using a cancer tissue sample.

9. The method of claim 2, wherein molecular subtype information is defined and trained based on an outcome of different treatments.

10. The method of claim 2, wherein molecular subtype information is defined and trained based on prognosis of cancer progression of a subject.

11. The method of claim 2, wherein molecular subtype information is defined and trained based on prognosis of cancer recurrence of a subject.

12. A method of training a machine learning model to detect a development of a resistance mechanism in a cancer, the method comprising:
obtaining, from a source, a set of training data, wherein the training data comprises methylation data derived from a targeted methylation sequencing assay;
annotating, subsequent to the obtaining, the set of training data by assigning a histologic label to each article of training data in the set;
applying the annotated set of training data to the machine learning model; and
optimizing, based on the applying, a pattern recognition capability of an algorithm associated with the machine learning model.

13. The method of claim 12, wherein the source is a plurality of training subjects.

14. The method of claim 12, wherein the histologic label is associated with one of: an adenocarcinoma and a small cell neuroendocrine carcinoma.

15. The method of claim 12, wherein the optimizing the pattern recognition capability of the algorithm comprises:
causing the machine learning model to:
A) determine whether minimal residual disease is present within a test set of methylation data; and
B) determine, responsive to determining that the minimal residual disease is present within the test set, whether at least a portion of cancer cells in the minimal residual disease have transformed from a first cancer type to a second cancer type as a result of the development of the resistance mechanism.

16. The method of claim 15, wherein the optimizing the pattern recognition capability of the algorithm comprises:
causing the machine learning model to:
C) suggest, responsive to determining that the minimal residual disease is present and the that at least a portion of cancer cells in the minimal residual disease have transformed from the first cancer type to the second cancer type, a treatment recommendation directed to the second cancer type.

17. A method of detecting a development of a resistance mechanism in a cancer undergoing a treatment using a trained machine learning model associated with a computer system, the method comprising:
receiving, from a biological sample associated with a test subject, methylation data derived from a targeted methylation sequencing assay;
applying, subsequent to the receiving, the methylation data to the trained machine learning model; and
receiving, subsequent to the applying, an output from the trained machine learning model, the output comprising:
A) a first indication of whether minimal residual disease is present within the test subject subsequent to administration of the treatment for the cancer; and
B) a second indication, responsive to the first indication providing a finding that the minimal residual disease is present within the test subject, of whether at least a portion of cancer cells in the minimal residual disease have transformed from a first cancer type to a second cancer type as a result of the development of the resistance mechanism.

18. The method of claim 17, wherein the resistance mechanism is transdifferentiation.

19. The method of claim 17, wherein the first cancer type is adenocarcinoma and wherein the second cancer type is a small cell neuroendocrine carcinoma.

20. The method of claim 17, wherein the output further comprises:
C) a third indication, responsive to the first indication providing the finding that the minimal residual disease is present within the test subject and the second indication providing another finding that the at least a portion of cancer cells in the minimal residual disease have transformed from the first cancer type to the second cancer type, and of a treatment recommendation directed to the second cancer type.

21. A method of training a machine learning model to generate a patient prognosis score, the method comprising:
obtaining, from a source, a set of training data, wherein the training data comprises methylation data derived from a targeted methylation sequencing assay;
annotating, subsequent to the obtaining, the set of training data by assigning known patient outcomes to each article of training data in the set;
applying the annotated set of training data to the machine learning model; and
optimizing, based on the applying, a patient prognosis prediction capability of an algorithm associated with the machine learning model.

22. A method of determining a final prognosis score for a test subject, the method comprising:
receiving, from a biological sample associated with a test subject, methylation data derived from a targeted methylation sequencing assay;
applying, subsequent to the receiving, the methylation data to a trained machine learning model;
receiving, subsequent to the applying, an output from the trained machine learning model, the output comprising a first prognosis score for the test subject;
identifying, based on the biological sample, a ctDNA score;
combining the first prognosis score and the ctDNA score together; and
generating, based on the combining, the final prognosis score for the test subject.

23. The method of claim 22, wherein the identifying the ctDNA score comprises:
ascertaining a tumor fraction value associated with the biological sample; and
computing the log 10 of the tumor fraction value.

24. The method of claim 23, wherein the combining comprises multiplying the log 10 of the tumor fraction value by the first prognosis score.

25. The method of claim 22, wherein the final prognosis score is a value between 0 and 1.
